(19) United States
(12) Patent Application Publication
(10) Pub. No.: US 2007/0026480 A1
(43) Pub. Date: Feb. 1, 2007

(54) Method and composition to evaluate cytochrome P450 2D6 isoenzyme activity using a breath test

(76) Inventors: Anil S. Modak, Methuen, MA (US); Yasuo Irie, Reading, MA (US); Yasuhisa Kurogi, North Andover, MA (US)

Correspondence Address: FOLEY & LARDNER LLP, 111 HUNTINGTON AVENUE, 26TH FLOOR, BOSTON, MA, 02199-7610, US

(21) Appl. No.: 11/404,965

(22) Filed: Apr. 14, 2006

Related U.S. Application Data
(60) Provisional application No. 60/671,784, filed on Apr. 16, 2005.

(51) Int. Cl. C12Q 1/26

(52) U.S. Cl. 435/25

(57) ABSTRACT

The present invention relates, generally to a method of determining and assessing cytochrome P450 2D6 isoenzyme (CYP2D6)-related metabolic capacity in an individual mammalian subject via a breath assay, by determining the relative amount of ¹³CO₂ exhaled by a the subject upon intravenous or oral administration of a ¹³C-labeled CYP2D6 substrate compound. The present invention is useful as an in vivo phenotype assay for evaluating CYP2D6-related activity using the metabolite ¹³CO₂ in expired breath and to determine the optimal dosage and timing of administration of CYP2D6 substrate compound.

### RELATED APPLICATION DATA

The present application claims priority to U.S. Provisional Patent Application No. 60/671,784 filed Apr. 16, 2005, which application is incorporated herein by reference to the extent permitted by law.

### FIELD OF THE INVENTION

The present invention relates, generally to a method of determining and assessing cytochrome P450 2D6-related (CYP2D6) metabolic capacity in an individual mammalian subject via a breath assay, by determining the relative amount of ¹³CO₂ exhaled by the subject upon intravenous or oral administration of a ¹³C-labeled CYP2D6 substrate compound. The present invention is useful as a non-invasive, in vivo assay for evaluating CYP2D6 enzyme activity in a subject using the metabolite ¹³CO₂ in expired breath, to phenotype individual subjects and to determine the selection, optimal dosage and timing of drug administration.

### BACKGROUND OF THE INVENTION

Many therapeutic compounds are effective in about 30-60% of patients with the same disease. (Lazarou, J. et al., J. Amer. Med. Assoc., 279: 1200-1205 (1998)). Further, a subset of these patients may suffer severe side effects which are among the leading cause of death in the United States and have an estimated $100 billion annual economic impact (Lazarou, J. et al., J. Amer. Med. Assoc., 279: 1200-1205 (1998)). Many studies have shown that patients differ in their pharmacological and toxicological reactions to drugs due, at least in part, to genetic polymorphisms which contribute to the relatively high degree of uncertainty inherent in the treatment of individuals with a drug. Single nucleotide polymorphisms (SNPs)—variations in DNA at a single base that are found in at least 1% of the population—are the most frequent polymorphisms in the human genome. Such subtle change(s) in the primary nucleotide sequence of a gene encoding a pharmaceutically-important protein may be manifested as significant variation in expression, structure and/or function of the protein.

Conventional medical approaches to diagnosis and treatment of disease is based on clinical data alone, or made in conjunction with a diagnostic test(s). Such traditional practices often lead to therapeutic choices that are not optimal for the efficacy of the prescribed drug therapy or to minimize the likelihood of side effects for an individual subject. Therapy specific diagnostics (a.k.a., theranostics) is an emerging medical technology field, which provides tests useful to diagnose a disease, choose the correct treatment regimen, and monitor a subject's response. That is, theranostics are useful to predict and assess drug response in individual subjects, i.e., individualized medicine. Theranostic tests are useful to select subjects for treatments that are particularly likely to benefit from the treatment or to provide an early and objective indication of treatment efficacy in individual subjects, so that the treatment can be altered with a minimum of delay. Theranostic tests may be developed in any suitable diagnostic testing format, which include, but is not limited to, e.g., non-invasive breath tests, immunohistochemical tests, clinical chemistry, immunoassay, cell-based technologies, and nucleic acid tests.

There is a need in the art for a reliable theranostic test to define a subject's phenotype or the drug metabolizing capacity to enable physicians to individualize therapy thereby avoiding potential drug related toxicity in poor metabolizers and increasing efficacy. Accordingly, there is a need in the art to develop new diagnostic assays useful to assess the metabolic activity of drug metabolizing enzymes such as the cytochrome P450 enzymes (CYPs) in order to determine individual optimized drug selection and dosages.

### SUMMARY OF THE INVENTION

The present invention relates to a diagnostic, noninvasive, in vivo phenotype test to evaluate CYP2D6 activity using a CYP2D6 substrate compound labeled with isotope incorporated at least at one specific position. The present invention utilizes the CYP2D6 enzyme-substrate interaction such that there is release of stable isotope-labeled CO₂ (e.g., ¹³CO₂) in the expired breath of a mammalian subject. The subsequent quantification of stable isotope-labeled CO₂ allows for the indirect determination of pharmacokinetics of the substrate and the evaluation of CYP2D6 enzyme activity (i.e., CYP2D6-related metabolic capacity).

In one aspect, the invention provides a preparation for determining CYP2D6-related metabolic capacity, comprising of an active ingredient a CYP2D6 substrate compound in which at least one of the carbon or oxygen atoms is labeled with an isotope, wherein the preparation is capable of producing isotope-labeled CO₂ after administration to a mammalian subject. In one embodiment of the preparation, the isotope is at least one isotope selected from the group consisting of: ¹³C; ¹⁴C; and ¹⁸O.

In another aspect, the invention provides a method for determining CYP2D6-related metabolic capacity, comprising the steps of administering to a mammalian subject, a preparation comprising of a CYP2D6 substrate compound in which at least one of the carbon or oxygen atoms is labeled with an isotope, wherein the preparation is capable of producing isotope-labeled CO₂ after administration to the mammalian subject, and measuring the excretion pattern of an isotope-labeled metabolite excreted from the body of the subject. In one embodiment of the method, the isotope-labeled metabolite is excreted from the body of a subject as isotope-labeled CO₂ in the expired air.

In one embodiment, the method of the invention is a method for determining CYP2D6-related metabolic capacity in a mammalian subject, comprising the steps of administering to the subject a preparation comprising of a CYP2D6 substrate compound in which at least one of the carbon or oxygen atoms is labeled with an isotope, wherein the preparation is capable of producing isotope-labeled CO₂ after administration to the mammalian subject, measuring the excretion pattern of an isotope-labeled metabolite excreted from the body of the subject, and assessing the obtained excretion pattern in the subject. In one embodiment, the method comprises the steps of administering to a mammalian subject a preparation comprising a CYP2D6 substrate compound in which at least one of the carbon or oxygen atoms is labeled with an isotope, wherein the preparation is capable of producing isotope-labeled CO₂ after administration to the mammalian subject, measuring the excretion pattern of isotope-labeled CO₂ in the expired air, and assessing the obtained excretion pattern of CO₂ in the subject. In one embodiment, the method comprises the steps of administering to a mammalian subject a preparation comprising of a CYP2D6 substrate compound in which at least one of the carbon or oxygen atoms is labeled with an isotope, wherein the preparation is capable of producing isotope-labeled CO₂ after administration to the mammalian subject, measuring the excretion pattern of an isotope-labeled metabolite, and comparing the obtained excretion pattern in the subject or a pharmacokinetic parameter obtained therefrom with the corresponding excretion pattern or parameter in a healthy subject with a normal CYP2D6-related metabolic capacity.

In one embodiment, the method of the invention is a method for determining the existence, nonexistence, or degree of CYP2D6-related metabolic disorder in a mammalian subject, comprising the steps of administering to the subject a preparation comprising a CYP2D6 substrate compound in which at least one of the carbon or oxygen atoms is labeled with an isotope, wherein the preparation is capable of producing isotope-labeled CO₂ after administration to a mammalian subject; measuring the excretion pattern of an isotope-labeled metabolite excreted from the body; and assessing the obtained excretion pattern in the subject.

In one embodiment, the method of the invention is a method for determining CYP2D6-related metabolic capacity, comprising of the steps of administering to a mammalian subject a preparation comprising of a CYP2D6 substrate compound in which at least one of the carbon or oxygen atoms is labeled with an isotope, wherein the preparation is capable of producing isotope-labeled CO₂ after administration to the mammalian subject; and measuring the excretion pattern of an isotope-labeled metabolite excreted from the body of the subject. In one embodiment of the method, the isotope-labeled metabolite is excreted from the body of the subject as isotope-labeled CO₂ in the expired air.

In one embodiment, the method of the invention is a method for selecting a prophylactic or therapeutic treatment for a subject, comprising: (a) determining the phenotype of the subject; (b) assigning the subject to a subject class based on the phenotype of the subject; and (c) selecting a prophylactic or therapeutic treatment based on the subject class, wherein the subject class comprises of two or more individuals who display a level of CYP2D6-related metabolic capacity that is at least about 10% lower than a reference standard level of CYP2D6-related metabolic capacity. In one embodiment of the method, the subject class comprises of two or more individuals who display a level of CYP2D6-related metabolic capacity that is at least about 10% higher than a reference standard level of CYP2D6-related metabolic capacity. In one embodiment of the method, the subject class comprises of two or more individuals who display a level of CYP2D6-related metabolic capacity within at least about 10% of a reference standard level of CYP2D6-related metabolic capacity. In one embodiment of the method, the treatment is selected from administering a drug, selecting a drug dosage, and selecting the timing of a drug administration.

In one embodiment, the method of the invention is a method for evaluating CYP2D6-related metabolic capacity, comprising the steps of: administering a ¹³C-labeled CYP2D6 substrate compound to a mammalian subject; measuring ¹³CO₂ exhaled by the subject; and determining CYP2D6-related metabolic capacity from the measured ¹³CO₂. In one embodiment of the method, the ¹³C-labeled CYP2D6 substrate compound is selected from the group consisting of: a ¹³C-labeled dextromethorphan; ¹³C-labeled tramadol; and ¹³C-labeled codeine. In one embodiment of the method, the ¹³C-labeled CYP2D6 substrate compound is administered non-invasively. In one embodiment, the ¹³C-labeled CYP2D6 substrate compound is administered intravenously or orally. In one embodiment of the method, the exhaled ¹³CO₂ is measured spectroscopically. In one embodiment of the method, the exhaled ¹³CO₂ is measured by infrared spectroscopy. In one embodiment of the invention, the exhaled ¹³CO₂ is measured with a mass analyzer. In one embodiment of the method, the exhaled ¹³CO₂ is measured over at least three time periods to generate a dose response curve, and the CYP2D6-related metabolic activity is determined from the area under the curve (AUC) or the percent dose recovery (PDR) or the delta over baseline (DOB) value at a particular timepoint or any other suitable pharmacokinetic parameter. In one embodiment of the method, the exhaled ¹³CO₂ is measured over at least two different dosages of the ¹³C-labeled CYP2D6 substrate compound. In one embodiment of the method, the exhaled ¹³CO₂ is measured during at least the following time points: t₀, a time prior to ingesting the ¹³C-labeled CYP2D6 substrate compound; t₁, a time after the ¹³C-labeled CYP2D6 substrate compound has been absorbed in the bloodstream of the subject; and t₂, a time during the first elimination phase. In one embodiment of the method, the CYP2D6-related metabolic capacity is determined from as the a slope of δ¹³CO₂ at time points t₁ and t₂ calculated according to the following equation: slope=[(δ¹³CO₂)₂−(δ¹³CO₂)₁]/(t₂−t₁)- wherein δ¹³CO₂ is the amount of exhaled ¹³CO₂. In one embodiment of the method, at least one CYP2D6 modulating agent is administered to the subject before administrating a ¹³C-labeled CYP2D6 substrate compound. In one embodiment of the method, CYP2D6 modulating agent is a CYP2D6 inhibitor. In one embodiment of the method, the CYP2D6 modulating agent is a CYP2D6 inducer.

In one embodiment, the method of the invention is a method of selecting a mammalian subject for inclusion in a clinical trial for determining the efficacy of a compound to prevent or treat a medical condition, comprising the steps of: (a) administering a ¹³C-labeled cytochrome P450 2D6 isoenzyme substrate compound to the subject; (b) measuring a metabolite excretion pattern of an isotope-labeled metabolite excreted from the body of the subject; and (c) comparing the obtained metabolite excretion pattern in the subject to a reference standard excretion pattern; (d) classifying the subject according to a metabolic phenotype selected from the group consisting of: poor metabolizer, intermediate metabolizer, extensive metabolizer, and ultrarapid metabolizer based on the obtained metabolite excretion pattern; and (e) selecting the subject classified as extensive metabolizer in step (d) for inclusion in the clinical trial.

In another aspect, the invention provides a kit comprising of: a ¹³C-labeled CYP2D6 substrate compound; and instructions provided with the substrate that describe how to determine ¹³C-labeled CYP2D6 substrate compound metabolism in a subject. In one embodiment of the kit, the kit further comprises of at least three breath collection bags. In one embodiment of the kit, the kit further comprises of a cytochrome P45 2D6 modulating agent.

### BRIEF DESCRIPTION OF THE DRAWINGS

The drawing figures depict preferred embodiments by way of example, not by way of limitations. In the figures, like reference numerals refer to the same or similar elements.

FIG. 1 shows graphs illustrating variance in CYP2D6 metabolism of dextromethorphan-O—¹³CH₃ (DXM-O—¹³CH₃) in human subjects. Panel A is a graph of the presence of ¹³CO₂ in breath samples expressed as delta over baseline (DOB) of two human subjects (i.e., Vlt 1 and Vlt 2) as a function of time (min). Panel B is a graph of the percentage dose recovery (PDR) of DXM-O—¹³CH₃ as ¹³CO₂ in breath samples of expired air observed in two human subjects. Volunteer 1 (Vlt 1; “♦” symbol) is an extensive metabolizer of DXM-O—¹³CH₃ who shows normal metabolism of DXM-O—¹³CH₃. Volunteer 2 (Vlt 2; “▴” symbol) is a poor metabolizer of DXM-O—¹³CH₃.

FIG. 2 shows graphs illustrating variance in CYP2D6 metabolism of tramadol-O—¹³CH₃ in human subjects. Panel A is a graph of the presence of ¹³CO₂ in breath samples expressed as DOB of two human subjects (i.e., Vlt 1 and Vlt 2) as a function of time (min). Panel B is a graph of the PDR of tramadol-O—¹³CH₃ as ¹³CO₂ in breath samples of expired air observed in two human subjects. Volunteer 1 (Vlt 1; “♦” symbol) is an extensive metabolizer of tramadol-O—¹³CH₃ who shows normal metabolism of tramadol-O—¹³CH₃. Volunteer 2 (Vlt 2; “▴” symbol) is a poor metabolizer of tramadol-O—¹³CH₃.

FIG. 3 shows graphs illustrating variance in CYP2D6 metabolism of dextromethorphan-O—¹³CH₃ (DXM-O—¹³CH₃) in human subjects. Panel A is a graph of the presence of ¹³CO₂ in breath samples expressed as delta over baseline (DOB) of three human subjects (i.e., Vlt 1, Vlt 2 and Vlt 3) as a function of time (min). Panel B is a graph of the percentage dose recovery (PDR) of DXM-O—¹³CH₃ as ¹³CO₂ in breath samples of expired air observed in three human subjects. Volunteer 1 (Vlt 1; “♦” symbol) is an extensive metabolizer of DXM-O—¹³CH₃ who shows normal metabolism of DXM-O—¹³CH₃. Volunteer 2 (Vlt 2; “▴” symbol) is a poor metabolizer of DXM-O—¹³CH₃. Volunteer 3 (Vlt 3; “▪” symbol) is an intermediate metabolizer of DXM-O—¹³CH₃.

### DETAILED DESCRIPTION OF THE INVENTION

It is to be appreciated that certain aspects, modes, embodiments, variations and features of the invention are described below in various levels of detail in order to provide a substantial understanding of the present invention. The present invention relates to a diagnostic, noninvasive, in vivo phenotype test to evaluate CYP2D6 activity (EC 1.14.14.1, a.k.a., debrisoquine 4-hydroxylase; CYPIID6), using a CYP2D6 substrate compound labeled with isotope incorporated at least at one specific position. The present invention utilizes the CYP2D6 enzyme-substrate interaction such that there is release of stable isotope-labeled CO₂ (e.g., ¹³CO₂) in the expired breath of a mammalian subject. The subsequent quantification of stable isotope-labeled CO₂ allows for the indirect determination of pharmacokinetics of the substrate and the evaluation of CYP2D6 enzyme activity (i.e., CYP2D6-related metabolic capacity). In one embodiment, the invention provides a breath test for evaluation of CYP2D6-related metabolic capacity based on the oral or i.v. administration of a stable isotope ¹³C-labeled CYP2D6 substrate compound and measurement of the ¹³CO₂/¹²CO₂ ratio in expired breath using commercially available instrumentation, e.g., mass or infrared (IR) spectrometers.

CYP2D6 catalyzes the hydroxylation of debrisoquine and accounts for approximately 2-5% of hepatic CYPs in mammals such as humans. CYP2D6 also metabolizes other compounds (See infra, Table 2). For example, psychotropic drugs (e.g., anti-depressants) that are CYP2D6 substrates include, but are not limited to, e.g., amitriptyline (Elavil); desipramine (Normramin); impramine; nortriptyline (Pamelor); trimipramine (Surmontil). Antipsychotic drugs that are CYP2D6 substrates include, but are not limited to, e.g., Perphenazine (Trilafon); Risperidone (Risperdal); Haloperidol (Haldol); and Thioridazine (Mellaril). Beta blockers that are CYP2D6 substrates include, but are not limited to, e.g., Metoprolol (Lopressor); Propranolol (Inderal); and Timolol. Analgesic drugs that are CYP2D6 substrates include, but are not limited to, e.g., Codeine; Dextromethorphan; Oxycodone; and Hydrocodone. Antiarrhythmic drugs that are CYP2D6 substrates include, but are not limited to, e.g., Encainide; Flecainide; Mexiletine; and Propafenone.

The CYPs that display functional polymorphism are quantitatively the most important Phase I drug transformation enzymes in mammals. Genetic variation of several members of this CYP gene superfamily have been extensively examined (Bertilsson et al., Br. J. Clin. Pharmacol., 53: 111-122 (2002)). CYP2D6 (Bertilsson et al., Br. J. Clin. Pharmacol., 53: 111-122 (2002)), CYP2C9 (Lee et al., Pharmacogenetics, 12: 251-263 (2002)), CYP2C19 (Xie et al., Pharmacogenetics, 9: 539-549 (1999)) and CYP2A6 (Raunio et al., Br. J. Clin. Pharmacol., 52: 357-363 (2001)) all exhibit functional polymorphisms that alter or deplete enzyme activity. The CYP2D6 gene locus is highly polymorphic with more than 75 allelic variants (See infra, Table 4). CYP2D6 polymorphism is a substantial clinical concern. Basically, CYP2D6 polymorphisms are genetic variations in oxidative drug metabolism characterized by three phenotypes; the poor metabolizer (PM) 0 functional alleles, the intermediate metabolizer (IM) 1 functional allele, the extensive metabolizer (EM) 2 functional alleles; and the ultrarapid metabolizer (UM) more than two functional alleles. Specifically, however, an expression pattern having lower oxidative drug metabolism than EM is classified as an intermediate metabolizer (IM), i.e., an expression pattern between EM and PM. These metabolizer categories, their clinical characteristics and suggested individualized therapy are detailed below in Table 1.

TABLE 1
Metabolizer Phenotypes, Clinical Characteristics and Individualized Therapy

| Metabolic | Rate | Plasma Drug | Clinical | Individualized |
|---|---|---|---|---|
| Phenotype | of Metabolism | Levels | Outcome | Therapy |
| Poor metabolizer | None | Toxic | Side effects | Decrease dose to |
| (PM) | | | | reduce toxicity |
| Intermediate | Reduced | High | Sometimes side | Normal dose |
| metabolizer (IM) | | | effects | |
| Extensive | Normal | Normal | Normal response | Normal dose |
| metabolizer (EM) | | | | |
| Ultrarapid | Rapid | Low | Reduced efficacy | Increase dose to |
| metabolizer (UM) | | | | increase efficacy |

As summarized in Table 1, dramatically reduced or deficient enzyme activity results in the PM phenotype and individuals with PM phenotypes are at risk for supra-therapeutic plasma concentrations of drugs primarily metabolized by the affected enzyme with conventional doses of the drug leading to toxic side effects. The CYP2D6 enzyme is deficient in up to 10% of the population (Pollock et al., Psychopharmacol. Bull., 31(2): 327-331 (1995). By contrast, CYP2D6-related therapeutic failure may also occur when patients are treated with conventional doses of drugs metabolized by enzyme pathways that exhibit enhanced activity due either to enzyme induction (Fuhr, Clin. Pharmacokinet., 38: 493-504 (2000)) or genetic alterations involving multiple gene copies organized in tandem in a single allele (Dahlen et al., Clin. Pharmacol. Ther., 63: 444-452 (1998); see generally, Table 1, EM and UM phenotypes). The method of the invention solves a need in the art for a rapid, noninvasive method useful to phenotype individuals in order to define therapeutic regimens in individual subjects that minimizes adverse drug reactions (ADRs) due either to CYP2D6 pharmacogenetic variability or the presence of adverse CYP2D6-related drug-drug interactions. In one embodiment of the method, the phenotype breath test is based on the administration of a suitably ¹³C stable isotope labeled (non-radioactive) substrate, and measurement of the ¹³CO₂/¹²CO₂ ratio in expired breath using commercially available instrumentation.

The diagnostic test of the present invention is advantageous as it is rapid and noninvasive, therefore placing less burden on the subject to give an accurate in vivo assessment of CYP2D6 enzyme activity both safely and without side effects. Accordingly, the various aspects of the present invention relate to preparations, diagnostic/theranostic methods and kits useful to identify individuals predisposed to disease or to classify individuals with regard to drug responsiveness, side effects, or optimal drug dose. Various particular embodiments that illustrate these aspects follow.

I. Definitions

As used herein, the term “clinical response” means any or all of the following: a quantitative measure of the response, no response, and adverse response (i.e., side effects).

As used herein, the term “CYP2D6 modulating agent” is any compound that alters (e.g., increases or decreases) the expression level or biological activity level of CYP2D6 polypeptide compared to the expression level or biological activity level of CYP2D6 polypeptide in the absence of the CYP2D6 modulating agent. CYP2D6 modulating agent can be a small molecule, polypeptide, carbohydrate, lipid, nucleotide, or combination thereof. The CYP2D6 modulating agent may be an organic compound or an inorganic compound.

As used herein, the term “effective amount” of a compound is a quantity sufficient to achieve a desired therapeutic and/or prophylactic effect, for example, an amount which results in the prevention of or a decrease in the symptoms associated with a disease that is being treated, e.g., depression and cardiac arrhythmia. The amount of compound administered to the subject will depend on the type and severity of the disease and on the characteristics of the individual, such as general health, age, sex, body weight and tolerance to drugs. It will also depend on the degree, severity and type of disease.

As used herein, the term “medical condition” includes, but is not limited to, any condition or disease manifested as one or more physical and/or psychological symptoms for which treatment is desirable, and includes previously and newly identified diseases and other disorders.

As used herein, the term “reference standard” means a threshold value or series of values derived from one or more subjects characterized by one or more biological characteristics, e.g., drug metabolic profile; drug metabolic rate, drug responsiveness, genotype, haplotype, phenotype, etc.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., monkey, rats, mice, guinea pigs and the like).

As used herein, the term “genotype” means an unphased 5′ to 3′ sequence of nucleotide pair(s) found at one or more polymorphic sites in a locus on a pair of homologous chromosomes in an individual. As used herein, genotype includes a full-genotype and/or a sub-genotype.

As used herein, the term “phenotype” means the expression of the genes present in an individual. This may be directly observable (e.g., eye color and hair color) or apparent only with specific tests (e.g., blood type, urine, saliva, and drug metabolizing capacity). Some phenotypes such as the blood groups are completely determined by heredity, while others are readily altered by environmental agents.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the administration of an agent or drug to a subject includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated by reference in their entirety and for all purposes to the same extent as if each individual publication, patent, or patent application was specifically and individually indicated to be incorporated by reference in its entirety for all purposes.

II. General

The mammalian liver plays a primary role in the metabolism of steroids, the detoxification of drugs and xenobiotics, and the activation of procarcinogens. The liver contains enzyme systems, e.g., the CYP system, that converts a variety of chemicals to more soluble products. The CYPs are among the major constituent proteins of the liver mixed function monooxygenases. There are a number of classes of CYPs which include the hepatic isoenzymes, e.g., CYP3As (40-60% hepatic P-450 isoenzymes); CYP2D6 (2-5% hepatic P-450 isoenzymes); CYP2As (<1% hepatic P-450 isoenzymes), CYP1A2, CYP2Cs. The action of CYPs facilitates the elimination of drugs and toxins from the body. Indeed, CYP action is often the rate-limiting step in pharmaceutical elimination. CYPs also play a role in the conversion of prodrugs to their biologically active metabolite(s).

The CYPs are quantitatively the most important Phase I drug biotransformation enzymes and genetic variation of several members of this gene superfamily has been extensively examined. In phase I metabolism of drugs and environmental pollutants CYPs often modify substrate with one or more water-soluble groups (such as hydroxyl), thereby rendering it vulnerable to attack by the phase II conjugating enzymes. The increased water-solubility of phase I and especially phase II products permits ready excretion. Consequently, factors that lessen the activity of CYPs usually prolong the effects of pharmaceuticals, whereas factors that increase CYP activity have the opposite effect.

CYP2D6 is involved in the biotransformation of more than 40 therapeutic drugs including several β-receptor antagonists, anti-arrhythmics, anti-depressants, and anti-psychotics and morphine derivatives as summarized below in Table 2. Isotopic labeling of the CYP2D6 substrates of Table 2 such that administration of the isotope-labeled substrate to a subject results in the release of stable isotopically labeled CO₂ yields compounds useful in the methods of the present invention.

TABLE 2
Summary of Select CYP2D6 Substrates

| CYP2D6 Substrate | Reference(s) |
|---|---|
| alprenolol | Eichelbaum, Fed. Proc., 43(8): 2298-2302 (1984); Otton et al., Life Sci., 34(1): 73-80 |
| | (1984) |
| amitriptyline | Mellstrom et al., Clin. Pharmacol. Ther., 39(4): 369-371 (1986); Baumann et al., J. Int. |
| | Clin. Psychopharmacol., 1(2): 102-112 (1986) |
| amphetamine | Dring et al., Biochem. J., (1970); 425-435; Smith RL, Xenobiotica, 16: 361-365 (1986) |
| aripiprazole | Swainston et al., Drugs, 64(15): 1715-36 (2004) |
| atomoxetine | Ring et al., Drug Meta. Dispos., 30(3): 319-23 (2002) |
| bufuralol | Boobis et al., Biochem. Pharmacol., 34(1): 65-71 (1985); Dayer et al., |
| | Biochem. Biophys. Res. Commun., 125(1): 374-380 (1984); Gut et al., FEBS Lett., |
| | 173(2): 287-290 (1984); Dayer et al., Biochem. Pharmacol., 36(23): 4145-4152 (1987) |
| carvedilol | |
| chlorpheniramine | |
| chlorpromazine | |
| clomipramine | Bertilsson et al., Acta Psychiatr. Scand., Suppl 1997; 391: 14-21 |
| codeine | Desmeules et al., Eur. J. Clin. Pharmacol., 1991; 41(1): 23-26 |
| debrisoquine | Sloan et al., Br. Med J., 2(6138): 655-657 (1978); Smith et al., Lancet, 1(8070): |
| | 943-944 (1978); Idle et al., Life Sci., 22(11): 979-983 (1978); Mahgoub et al., Lancet, |
| | 2(8038): 584-586 (1977) |
| desipramine | Dahl et al., Eur. J. Clin. Pharmacol., 44: 445-45 (1993) |
| dexfenfluramine | Gross et al., Br. J. Clin. Pharmacol., 41: 311-317 1996 |
| dextromethorphan | Perault et al., Therapie, 46(1): 1-3 (1991) |
| doxepin | Szewczuk-Boguslawska et al., Pol. J. Pharmacol., 56(4): 491-4 (2004) |
| duloxetine | Skinner et al., Clin Pharmacol Ther., 73(3): 170-7 (2003) |
| encainide | Funck-Brentano et al., J. Pharmacol. Exp. Ther., 249(1): 134-42 (1989) |
| flecainide | Funck-Brentano et al., Clin. Pharmacol. Ther., 55(3): 256-269 (1994) |
| fluoxetine | Hamelin et al., Clin. Pharmacol. Ther., 60: 512-521 (1996) |
| fluvoxamine | Carillo et al., Clin. Pharmacol. Ther., 60: 183-190 (1996); Hamelin et al., Drug Metab |
| | Dispos., 26(6): 536-9 (1998) |
| haloperidol | Lierena et al., Ther. Drug. Monit., 14: 261-264 (1992) |
| imipramine | BrØsen et al., Clin. Pharmacol. Ther., 49(6): 609-617 (1991) |
| lidocaine | |
| metoclopramide | |
| methoxyamphetamine | |
| S-metoprolol | Ellis et al., Biochem. J., 316(Pt 2): 647-654 (1996); Lewis et al., Br. J. Clin. |
| | Pharmacol., 31(4): 391-398 (1991); Jonkers et al., J. Pharmacol. Exp. Ther., 256(3): |
| | 959-966 (1991); Lennard et al., Xenobiotica, 16(5): 435-447 (1986); Leemann et al., |
| | Eur. J. Clin. Pharmacol., 29(6): 739-741 (1986); McGourty et al., Br. J. Clin. |
| | Pharmacol., 20(6): 555-566 (1985); Lennard et al., Clin. Pharmacol. Ther., 34(6): |
| | 732-737 (1983); Lennard et al., N Engl J Med, 16; 307(25): 1558-1560 (1982); Lennard |
| | et al., Br. J. Clin. Pharmacol., 14(2): 301-303 (1982). |
| mexiletine | |
| minaprine | Marre et al., Drug Metab Dispos., 20(2): 316-321 (1992) |
| Nortriptyline | |
| Ondansetron | Carillo et al., Clin. Pharmacol. Ther., 60: 183-190 (1996) |
| Paroxetine | |
| Perhexiline | |
| perphenazine | Dahl-Puustinen et al., Clin. Pharmacol. Ther., 46(1): 78-81 (1989); Linnet et al., Clin. |
| | Pharmacol. Ther., 60: 41-47 (1996); Skjelbo and Brosen, Br. J. Clin. Pharmacol., 34: |
| | 256-261 (1992) |
| Phenacetin | |
| phenformin | |
| propafenone | Lee et al., N. Eng. J. Med., 332(25): 1764-1768 (1990) |
| propanolol | |
| quanoxan | |
| risperidone | Huang et al., Clin. Pharmacol. Ther., 54(3): 257-268 (1993) |
| sparteine | Bertilsson et al., Eur. J. Clin. Pharmacol., 17(2): 153-155 (1980); Eichelbaum et al., |
| | Eur. J. Clin. Pharmacol., 16(3): 189-194 (1979); Eichelbaum et al., Eur. J. Clin. |
| | Pharmacol., 16(3): 183-187 (1979); Spannbrucker et al., Verh. Dtsch. Ges. Inn. Med., |
| | 84: 1125-1127 (1978; German) |
| tamoxifen | Daniels et al., Br. J. Clin. Pharmacol., 33: 153P (1992); Stearns et al., J. Natl. Cancer |
| | Inst., 95(23): 1734-5 (2003) |
| thioridazine | von Bahr et al., Clin. Pharmacol. Ther., 49: 234-240 (1991) |
| timolol | Edeki et al., JAMA., 274(20): 1611-1613 (1995); Huupponen et al., J. Ocul. |
| | Pharmacol., 7(2): 183-187 (1991); al-Sereiti et al., Int. J. Clin. Pharmacol. Res., 10(6): |
| | 339-345 (1990); Salminen et al., Int. Ophthalmol., 13(1-2): 91-93 (1989); Lennard et |
| | al., Xenobiotica, 16(5): 435-447 (1986); McGourty et al., Clin. Pharmacol. Ther., |
| | 38(4): 409-413 (1985); Lewis et al., Br J Clin Pharmacol. 19(3): 329-333 (1985); |
| | Lennard and Parkin, J. Chromatogr., 338(1): 249-252 (1985); Smith RL, Eur. J. Clin. |
| | Pharmacol., 28 Suppl: 77-84 (1985) |
| tramadol | Dayer et al., Drugs, 53 Suppl 2: 18-24 (1997); Borlak et al., 52(11): 1439-43 (2003) |
| venlafaxine | Fogelman et al., Neuropsychopharmacology, 20(5): 480-90 (1999) |

Select agents can induce or inhibit CYP2D6 activity (i.e., CYP2D6 modulating agents). CYP modulating agents are useful in the methods of the present invention. Compounds known to inhibit CYP2D6 are summarized below in Table 3. The compounds include, psychotropic drugs that are CYP2D6 inhibitors include, e.g., Fluoxetine (Prozac). The antipsychotic drugs Haloperidol (Haldol); and Thioridazine (Mellaril) can also inhibit CYP2D6 activity. Analgesic drugs can inhibit CYP2D6, e.g., Celecoxib (Celebrex). Antiarrhythmic drugs can also inhibit CYP2D6, e.g., Amiodarone and Quinidine. Other drugs that inhibit CYP2D6 include, e.g., Cimetidine and Diphenhydramine. Inhibitors of CYP2D6 are useful as CYP2D6 modulating agents in the methods of the present invention.

TABLE 3
Summary of Select CYP2D6 Inhibitors

| CYP2D6 Inhibitor | Reference(s) |
|---|---|
| amiodarone | |
| buproprion | |
| celecoxib | |
| chlorpheniramine | |
| chlorpromazine | |
| cimetidine | Knodell et al., Gastroenterology, 101: 1680-1691 (1991) |
| citalopram | Clin Pharmacokinet., 32 Suppl 1: 1-21 (1997) |
| clomipramine | Lamard et al., Ann. Med. Psychol. (Paris), 153(2): 140-143 (1995) |
| cocaine | Tyndale et al., Mol. Pharmacol., 40: 63-68 (1991) |
| doxorubicin | Le Guellec et al., Cancer Chemother. Pharmacol., 32: 491-495 (1993) |
| escitalopram | |
| fluoxetine | |
| halofantrine | |
| levomepromazine | |
| methadone | Wu et al., Br. J. Clin. Pharmacol., 35(1): 30-34 (1993) |
| moclobemide | Gram et al., Clin. Pharmacol. Ther., 57(6): 670-677 (1995) |
| paroxetine | Brosen et al., Eur. J. Clin. Pharmacol., 44: 349-355 (1993) |
| quinidine | |
| ranitidine | |
| reduced haloperidol | Tyndale et al., Br. J. Clin. Pharmacol., 31: 655-660 (1991) |
| ritonavir | Kumar et al., J. Pharmacol. Exp. Ther., 277(1): 423-431 (1996) |
| sertraline | |
| terbinafine | |

Drugs that induce CYP2D6 include, e.g., Ritonavir; Amiodarone; Quinidine; Paroxetine; Cimetidine; Fluoxetine; dexamethasone; and Rifampin (Eichelbaum et al., Br. J. Clin. Pharmacol., 22:49-53 (1986); Eichelbaum et al., Xenobiotica, 16(5):465-481 (1986)). Inducers of CYP2D6 are useful as CYP2D6 modulating agents in the methods of the present invention.

III. CYP2D6 Polymorphism and Clinical Response

Genetic polymorphism of CYPs results in subpopulations of individual subjects that are distinct in their ability to perform particular drug biotransformation reactions. These phenotypic distinctions have important implications for the selection of drugs. For example, a drug that is safe when administered to a majority of subjects (e.g., human subjects) may cause intolerable side effects in an individual subject suffering from a defect in a CYP enzyme required for detoxification of the drug. Alternatively, a drug that is effective in most subjects may be ineffective in a particular subpopulation of subjects because of the lack of a particular CYP enzyme required for conversion of the drug to a metabolically active form. Accordingly, it is important for both drug development and clinical use to screen drugs to determine which CYPs are required for activation and/or detoxification of the drug.

It is also important to identify those individuals who are deficient in a particular CYP. This type of information has been used to advantage in the past for developing genetic assays that predict phenotype and thus predict an individual's ability to metabolize a given drug. This Information is of particular value in determining the likely side effects and therapeutic failures of various drugs. Routine phenotyping is useful for certain categories (e.g., PM, IM, EM and UM subjects) of subjects in need thereof. Such phenotyping is also useful in the selection (inclusion/exclusion) of candidate subjects for enrolled in drug clinical trails.

As noted above, more than 75 allelic variants of the CYP2D6 gene locus have been identified as summarized below in Table 4.

TABLE 4
CYP2D6 Allelic Variants

| | | | | Enzyme activity | |
|---|---|---|---|---|---|
| Allele | Protein | Nucleotide changes, gene | Effect | In vivo | In vitro |
| CYP2D6*1A | CYP2D6.1 | None | | Normal | Normal |
| (a.k.a., wild | | | | | |
| type) | | | | | |
| CYP2D6*1B | CYP2D6.1 | 3828G>A | | Normal | |
| | | | | (d, s) | |
| CYP2D6*1C | CYP2D6.1 | 1978C>T | | Normal | |
| (a.k.a., M4) | | | | (s) | |
| CYP2D6*1D | CYP2D6.1 | 2575C>A | | | |
| (a.k.a., M5) | | | | | |
| CYP2D6*1E | CYP2D6.1 | 1869T>C | | | |
| CYP2D6*1XN | CYP2D6.1 | | N active | Incr | |
| | | | genes | | |
| CYP2D6*2A | CYP2D6.2 | −1584C>G; −1235A>G; | R296C; S486T | Normal | |
| (a.k.a, | | −740C>T; −678G>A; | | (dx, d, s) | |
| CYP2D6L) | | CYP2D7 gene conversion in | | | |
| | | intron 1; 1661G>C; | | | |
| | | 2850C>T; 4180G>C | | | |
| CYP2D6*2B | CYP2D6.2 | 1039C>T; 1661G>C; | R296C; S486T | | |
| | | 2850C>T; 4180G>C | | | |
| CYP2D6*2C | CYP2D6.2 | 1661G>C; 2470T>C; | R296C; S486T | | |
| | | 2850C>T; 4180G>C | | | |
| CYP2D6*2 | CYP2D6.2 | 2850C>T; 4180G>C | R296C; S486T | | |
| (a.k.a., M10) | | | | | |
| CYP2D6*2E | CYP2D6.2 | 997C>G; 1661G>C; | R296C; S486T | | |
| (a.k.a., M12) | | 2850C>T; 4180G>C | | | |
| CYP2D6*2F | CYP2D6.2 | 1661G>C; 1724C>T; | R296C; S486T | | |
| (a.k.a., M14) | | 2850C>T; 4180G>C | | | |
| CYP2D6*2G | CYP2D6.2 | 1661G>C; 2470T>C; | R296C; S486T | | |
| (a.k.a., M16) | | 2575C>A; 2850C>T; | | | |
| | | 4180G>C | | | |
| CYP2D6*2H | CYP2D6.2 | 1661G>C; 2480C>T; | R296C; S486T | | |
| (a.k.a., M17) | | 2850C>T; 4180G>C | | | |
| CYP2D6*2J | CYP2D6.2 | 1661G>C; 2850C>T; | R296C; S486T | | |
| (a.k.a., M18) | | 2939G>A; 4180G>C | | | |
| CYP2D6*2K | CYP2D6.2 | 1661G>C; 2850C>T; | R296C; S486T | | |
| (a.k.a., M21) | | 4115C>T; 4180G>C | | | |
| CYP2D6*2XN | CYP2D6.2 | 1661G>C; | R296C; S486T | Incr | |
| (N = 2, 3, 4, 5 | | 2850C>T; 4180G>C | N active genes | (d) | |
| or 13) | | | | | |
| CYP2D6*3A | | 2549A>del | Frameshift | None | None |
| (a.k.a., | | | | (d, s) | (b) |
| CYP2D6A) | | | | | |
| CYP2D6*3B | | 1749A>G; 2549A>del | N166D; | | |
| | | | frameshift | | |
| CYP2D6*4A | | 100C>T; 974C>A; 984A>G; _997C>G; | P34S; L91M; | None | None |
| (a.k.a., | | 1661G>C; | H94R; Splicing | (d, s) | (b) |
| CYP2D6B) | | 1846G>A; 4180G>C | defect; S486T | | |
| CYP2D6*4B | | 100C>T; 974C>A; 984A>G; | P34S; L91M; | None | None |
| (a.k.a., | | 997C>G; 1846G>A; | H94R; Splicing | (d, s) | (b) |
| CYP2D6B) | | 4180G>C | defect; S486T | | |
| CYP2D6*4C | | 100C>T; 1661G>C; | P34S; Splicing | None | |
| (a.k.a., K29-1) | | 1846G>A; 3887T>C; | defect; L421P; | | |
| | | 4180G>C | S486T | | |
| CYP2D6*4D | | 100C>T; 1039C>T; | P34S; Splicing | None (dx) | |
| | | 1661G>C; 1846G>A; | defect; S486T | | |
| | | 4180G>C | | | |
| CYP2D6*4E | | 100C>T; 1661G>C; | P34S; Splicing | | |
| | | 1846G>A; 4180G>C | defect; S486T | | |
| CYP2D6*4F | | 100C>T; 974C>A; 984A>G; | P34S; L91M; | | |
| | | 997C>G; 1661G>C; | H94R; Splicing | | |
| | | 1846G>A; | defect; R173C; | | |
| | | 1858C>T; 4180G>C | S486T | | |
| CYP2D6*4G | | 100C>T; 974C>A; 984A>G; | P34S; L91M; | | |
| | | 997C>G; 1661G>C; | H94R; Splicing | | |
| | | 1846G>A; 2938C>T; | defect; P325L; | | |
| | | 4180G>C | S486T | | |
| CYP2D6*4H | | 100C>T; 974C>A; 984A>G; | P34S; L91M; | | |
| | | 997C>G; 1661G>C; | H94R; Splicing | | |
| | | 1846G>A; 3877G>C; | defect; E418Q; | | |
| | | 4180G>C | S486T | | |
| CYP2D6*4J | | 100C>T; 974C>A; 984A>G; | P34S; L91M; | | |
| | | 997C>G; 1661G>C; | H94R; Splicing | | |
| | | 1846G>A | defect | | |
| CYP2D6*4K | | 100C>T; 1661G>C; | P34S; Splicing | None | |
| | | 1846G>A; 2850C>T; | defect; R296C; | | |
| | | 4180G>C | S486T | | |
| CYP2D6*4L | | 100C>T; 997C>G; 1661G>C; | P34S; Splicing | | |
| | | 1846G>A; 4180G>C | defect; S486T | | |
| CYP2D6*4X2 | | | | None | |
| CYP2D6*5 | | CYP2D6 deleted | CYP2D6 | None | |
| (a.k.a., | | | deleted | (d, s) | |
| CYP2D6D) | | | | | |
| CYP2D6*6A | | 1707T>del | Frameshift | None | |
| (a.k.a., | | | | (d, dx) | |
| CYP2D6T) | | | | | |
| CYP2D6*6B | | 1707T>del; 1976G>A | Frameshift; | None | |
| | | | G212E | (s, d) | |
| CYP2D6*6C | | 1707T>del; 1976G>A; | Frameshift; | None (s) | |
| | | 4180G>C | G212E; S486T | | |
| CYP2D6*6D | | 1707T>del; 3288G>A | Frameshift; | | |
| | | | G373S | | |
| CYP2D6*7 | CYP2D6.7 | 2935A>C | H324P | None | |
| (a.k.a., | | | | (s) | |
| CYP2D6E) | | | | | |
| CYP2D6*8 | | 1661G>C; 1758G>T; | Stop codon; | None | |
| (a.k.a., | | 2850C>T; 4180G>C | R296C; S486T | (d, s) | |
| CYP2D6G) | | | | | |
| CYP2D6*9 | CYP2D6.9 | 2613-2615delAGA | K281del | Decr | Decr |
| (a.k.a., | | | | (b, s, d) | (b, s, d) |
| CYP2D6C) | | | | | |
| CYP2D6*10A | CYP2D6.10 | 100C>T; 1661G>C; | P34S; S486T | Decr | |
| (a.k.a., | | 4180G>C | | (s) | |
| CYP2D6J) | | | | | |
| CYP2D6*10B | CYP2D6.10 | −1426C>T; −1236/−1237insAA; | P34S; S486T | Decr | Decr |
| (a.k.a., | | −1235A>G; | | (d) | (b) |
| CYP2D6Ch1) | | −1000G>A; 100C>T; | | | |
| | | 1039C>T; 1661G>C; | | | |
| | | 4180G>C | | | |
| CYP2D6*10C | | | | | |
| CYP2D6*10D | CYP2D6.10 | 100C>T; 1039C>T; | P34S; S486T | | |
| | | 1661G>C; 4180G>C, | | | |
| | | CYP2D7-like 3′-flanking | | | |
| | | region | | | |
| CYP2D6*10X2 | CYP2D6.10 | | | Decr | |
| | | | | (dx) | |
| CYP2D6*11 | | 883G>C; 1661G>C; | Splicing defect; | None | |
| (a.k.a., | | 2850C>T; 4180G>C | R296C; S486T | (s) | |
| CYP2D6F) | | | | | |
| CYP2D6*12 | CYP2D6.12 | 124G>A; 1661G>C; | G42R;; R296C; | None | |
| | | 2850C>T; 4180G>C | S486T | (s) | |
| CYP2D6*13 | | CYP2D7P/CYP2D6 hybrid. | Frameshift | None | |
| | | Exon 1 CYP2D7, exons 2-9 | | (dx) | |
| | | CYP2D6. | | | |
| CYP2D6*14A | CYP2D6.14A | 100C>T; 1758G>A; | P34S; G169R; | None | |
| | | 2850C>T; 4180G>C | R296C; S486T | (d) | |
| CYP2D6*14B | CYP2D6.14B | intron 1 | G169R; R296C; | | |
| | | conversion with CYP2D7 | S486T | | |
| | | (214-245); 1661G>C; | | | |
| | | 1758G>A; 2850C>T; | | | |
| | | 4180G>C | | | |
| CYP2D6*15 | | 138insT | Frameshift | None | |
| | | | | (d, dx) | |
| CYP2D6*1 | | CYP2D7P/CYP2D6 hybrid. | Frameshift | None | |
| (a.k.a., | | Exons 1-7 CYP2D7P-related, | | (d) | |
| CYP2D6D2) | | exons 8-9 CYP2D6. | | | |
| CYP2D6*17 | CYP2D6.17 | 1023C>T; 2850C>T; | T107I; R296C; | Decr | Decr |
| (a.k.a., | | 4180G>C | S486T | (d) | (b) |
| CYP2D6Z) | | | | | |
| CYP2D6*18 | CYP2D6.18 | 4125-4133insGTGCCCACT | 468-470VPT ins | None (s) | Decr (b) |
| (a.k.a., | | | | | |
| CYP2D6(J9)) | | | | | |
| CYP2D6*19 | | 1661G>C; | Frameshift; | None | |
| | | 2539-2542delAACT; | R296C; S486T | | |
| | | 2850C>T; 4180G>C | | | |
| CYP2D6*20 | | 1661G>C; 1973insG; | Frameshift; | None (m) | |
| | | 1978C>T; 1979T>C; | L213S; R296C; | | |
| | | 2850C>T; 4180G>C | S486T | | |
| CYP2D6*21A | | −1584C>G; −1426C>T; −1258insAAAAA; | Frameshift; | None | |
| | | −1235A>G; −740C>T; | R296C; S486T | | |
| | | −678G>A; −629A>G; | | | |
| | | 214G>C; 221C>A; 223C>G; | | | |
| | | 227T>C; 310G>T; 601delC; | | | |
| | | 1661G>C; 2573insC; | | | |
| | | 2850C>T; 3584G>A; | | | |
| | | 4180G>C; | | | |
| | | 4653_4655delACA | | | |
| CYP2D6*21B | | −1584C>G; −1235A>G; −740C>T; | Frameshift; | None | |
| | | −678G>A; intron 1 | R296C; S486T | | |
| | | conversion with CYP2D7 | | | |
| | | (214-245); 1661G>C; | | | |
| | | 2573insC; 2850C>T; | | | |
| | | 4180G>C | | | |
| CYP2D6*22 | CYP2D6.22 | 82C>T | R28C | | |
| (a.k.a., M2) | | | | | |
| CYP2D6*23 | CYP2D6.23 | 957C>T | A85V | | |
| (a.k.a., M3) | | | | | |
| CYP2D6*24 | CYP2D6.24 | 2853A>C | I297L | | |
| (a.k.a., M6) | | | | | |
| CYP2D6*25 | CYP2D6.25 | 3198C>G | R343G | | |
| (a.k.a., M7) | | | | | |
| CYP2D6*26 | CYP2D6.26 | 3277T>C | I369T | | |
| (a.k.a., M8) | | | | | |
| CYP2D6*2 | CYP2D6.27 | 3853G>A | E410K | | |
| (a.k.a., M9) | | | | | |
| CYP2D6*28 | CYP2D6.28 | 19G>A; 1661G>C; | V7M; Q151E; | | |
| (a.k.a., M11) | | 1704C>G; 2850C>T; | R296C; S486T | | |
| | | 4180G>C | | | |
| CYP2D6*29 | CYP2D6.29 | 1659G>A; 1661G>C; | V136M; R296C; | | |
| (a.k.a., M13) | | 2850C>T; 3183G>A; | V338M; S486T | | |
| | | 4180G>C | | | |
| CYP2D6*30 | CYP2D6.30 | 1661G>C; 1863 ins 9bp rep; | 172-174FRP | | |
| (a.k.a., M15) | | 2850C>T; 4180G>C | rep; R296C; | | |
| | | | S486T | | |
| CYP2D6*31 | CYP2D6.31 | 1661G>C; 2850C>T; | R296C; R440H; | | |
| (a.k.a., M20) | | 4042G>A; 4180G>C | S486T | | |
| CYP2D6*32 | CYP2D6.32 | 1661G>C; 2850C>T; | R296C; E410K; | | |
| (a.k.a., M19) | | 3853G>A; 4180G>C | S486T | | |
| CYP2D6*33 | CYP2D6.33 | 2483G>T | A237S | Normal | |
| (a.k.a., | | | | (s) | |
| CYP2D6*1C) | | | | | |
| CYP2D6*34 | CYP2D6.34 | 2850C>T | R296C | | |
| (a.k.a., | | | | | |
| CYP2D6*1D) | | | | | |
| CYP2D6*35 | CYP2D6.35 | −1584C>G; 31G>A; | V11M; R296C; | Normal | |
| (a.k.a., | | 1661G>C; 2850C>T; | S486T | (s) | |
| CYP2D6*2B) | | 4180G>C | | | |
| CYP2D6*35X2 | CYP2D6.35 | 31G>A; 1661G>C; 2850C>T; | V11M; R296C; | Incr | |
| | | 4180G>C | S486T | | |
| CYP2D6*36 | CYP2D6.36 | −1426C>T; −1236/−1237insA; | P34S; P469A; | Decr | Decr |
| (a.k.a., | | −1235A>G; | T470A; H478S; | (d) | (b) |
| CYP2D6Ch2) | | −1000G>A; 100C>T; | G479A; F481V; | | |
| | | 1039C>T; 1661G>C; | A482S; S486T | | |
| | | 4180G>C; gene conversion | | | |
| | | to CYP2D7 in exon 9 | | | |
| CYP2D6*37 | CYP2D6.37 | 100C>T; 1039C>T; | P34S; R201H; | | |
| (a.k.a, | | 1661G>C; 1943G>A; | S486T | | |
| CYP2D6*10D) | | 4180G>C; | | | |
| CYP2D6*38 | | 2587-2590delGACT | Frameshift | None | |
| CYP2D6*39 | CYP2D6.39 | 1661G>C; 4180G>C | S486T | | |
| CYP2D6*40 | CYP2D6.40 | 1023C>T; 1661G>C; 1863ins | T107I; | None (dx) | |
| | | (TTT CGC CCC)2; 2850C>T; | 172-174(FRP)3; | | |
| | | 4180G>C | R296C; S486T | | |
| CYP2D6*41A | CYP2D6.2 | −1584C; −1235A>G; −740C>T; | R296C; S486T | Decr (s) | |
| | | −678G>A; CYP2D7 gene | | | |
| | | conversion in intron 1; | | | |
| | | 1661G>C; 2850C>T; | | | |
| | | 2988G>A; 4180G>C | | | |
| CYP2D6*41B | CYP2D6.2 | −1548C; −1298G>A; −1235A>G; | R296C; S486T | | |
| | | −740C>T; 310G>T; | | | |
| | | 746C>G; 843T>G; 1513C>T; | | | |
| | | 1661G>C; 1757C>T; | | | |
| | | 2850C>T; 3384A>C; | | | |
| | | 3584G>A; 3790C>T; | | | |
| | | 4180G>C; 4656-58delACA; | | | |
| | | 4722T>G | | | |
| CYP2D6*42 | CYP2D6.42 | −1584C; 1661G>C; | R296C; | None | |
| | | 2850C>T; 3259insGT; | Frameshift | (dx) | |
| | | 4180G>C | S486T | | |
| CYP2D6*43 | CYP2D6.43 | 77G>A | R26H | | |
| (a.k.a., M1) | | | | | |
| CYP2D6*44 | CYP2D6.44 | 82C>T; 2950G>C | Splicing defect | None | |
| CYP2D6*45A | CYP2D6.45 | −1600GA>TT; −1584C; −1237-36delAA; | E155K; R296C; | | |
| | | −1093insA; −1011T>C; | S486T | | |
| | | 310G>T; 746C>G; | | | |
| | | 843T>G; 1661G>C; | | | |
| | | 1716G>A; 2129A>C; | | | |
| | | 2575C>A; 2661G>A; | | | |
| | | 2850C>T; 3254T>C; | | | |
| | | 3384A>C; 3584G>A; | | | |
| | | 3790C>T; 4180G>C; | | | |
| | | 4656-58delACA; 4722T>G | | | |
| CYP2D6*45B | CYP2D6.45 | −1584C; −1543G>A; −1298G>A; | E155K; R296C; | | |
| | | −1235A>G; −1093insA; −740C>T; | S486T | | |
| | | −693-90delTGTG; | | | |
| | | 310G>T; 746C>G; 843T>G; | | | |
| | | 1661G>C; 1716G>A; | | | |
| | | 2575C>A; 2661G>A; | | | |
| | | 2850C>T; 3254T>C; | | | |
| | | 3384A>C; 3584G>A; | | | |
| | | 3790C>T; 4180G>C; | | | |
| | | 4656-58delACA; 4722T>G | | | |
| CYP2D6*46 | CYP2D6.46 | −1584C; −1543G>A; −1298G>A; | R26H; E155K; | | |
| | | −1235A>G; −740C>T; | R296C; S486T | | |
| | | 77G>A; 310G>T; 746C>G; | | | |
| | | 843T>G; 1661G>C; | | | |
| | | 1716G>A; 2575C>A; | | | |
| | | 2661G>A; 2850C>T; | | | |
| | | 3030G>G/A*; 3254T>C; | | | |
| | | 3384A>C; 3491G>A; | | | |
| | | 3584G>A; 3790C>T; | | | |
| | | 4180G>C; 4656-58delACA; | | | |
| | | 4722T>G | | | |
| | | *Both haplotypes have been | | | |
| | | described (Gaedigk et al. | | | |
| | | 2005) | | | |
| CYP2D6*47 | CYP2D6.47 | −1426C>T; −1235A>G; −1000G>A; | R25W; P34S; | | |
| | | 73C<T; 100C>T; | S486T | | |
| | | 1039C>T; 1661G>C; | | | |
| | | 4180G>C | | | |
| CYP2D6*48 | CYP2D6.48 | 972C>T | A90V | | |
| CYP2D6*49 | CYP2D6.49 | −1426C>T; −1235A>G; −1000G>A; | P34S; F120I; | | |
| | | 100C>T; 1039C>T; | S486T | | |
| | | 1611T>A; 1661G>C; | | | |
| | | 4180G>C | | | |
| CYP2D6*50 | CYP2D6.50 | 1720A>C | E156A | | |
| CYP2D6*51 | CYP2D6.51 | −1584C>G; −1235A>G; −740C>T; | R296C; E334A; | | |
| | | −678G>A; CYP2D7 | S486T | | |
| | | gene conversion in intron 1; | | | |
| | | 1661G>C; 2850C>T; | | | |
| | | 3172A>C; 4180G>C | | | |

In the columns showing Enzyme activity in Table 4, Bufuralol is designated by the letter “b”; Debrisoquine is designated by the letter “d”; Dextromethorphan is designated by the letters “dx”; and Sparteine is designated by the letter “s”.

As detailed in Table 4, individual alleles are designated by the gene name (CYP2D6) followed by an asterisk and an Arabic number, e.g., CYP2D6*1A designates, by convention, the fully functional wild-type allele. Allelic variants are the consequence of point mutations, single base pair deletions or additions, gene rearrangements or deletion of the entire gene that can result in a reduction or complete loss of activity. Inheritance of two recessive loss-of-function alleles results in the PM phenotype, which is found in about 5 to 10% of Caucasians and about 1 to 2% of Asian subjects. In Caucasians, the *3, *4, *5 and *6 alleles are the most common loss-of-function alleles and account for approximately 98% of poor metabolizer phenotype. Gaedigk et al., Pharmacogenetics, 9: 669-682 (1999). In contrast, CYP2D6 activity on a population basis is lower in Asian and African American populations due to a lower frequency of non-functional alleles (*3, *4, *5 and *6) and a relatively high frequency of population-selective alleles that are associated with decreased activity relative to the wild-type CYP2D6*1 allele. For example, the CYP2D6*10 allele occurs at a frequency of approximately 50% in Asians (Johansson et al., Mol. Pharmacol., 46: 452-459 (1994); Bertilsson, Clin. Pharmacokin., 29: 192-209 (1995)) while CYP2D6*17 and CYP2D6*29 occur at relatively high frequencies in subjects of black African origin (Gaedigk et al., Clin. Pharmacol. Ther., 72: 76-89 (2002); Masimirembwa et al., Br. J. Clin. Pharmacol., 42: 713-719 (1996)).

The clinical consequences of variable CYP2D6 activity are primarily related to reduced clearance of drug substrates and have been recently reviewed (Bertilsson et al., Br. J. Clin. Pharmacol., 53: 111-122 (2002)). In essence, drug clearance is decreased and consequently, plasma drug concentrations are increased with the attendant risk of ADRs in individuals who are PMs by genotype or functionally PMs due to other factors, e.g., a drug interaction.

Stable isotope tracer probes are ideal tools for the non-invasive kinetic assessment of the in vivo metabolism of drugs to classify the CYP2D6 metabolic status of individual subjects especially in the pediatric population. One important consequence of inter-individual variability in drug disposition and response is the risk of ADRs. In the case of pharmacogenetic variability, genotypic and phenotypic characterization of individual patients or patient populations is useful to predict enzyme activity and to optimize drug safety and efficacy. It could also play a significant role in the selection (inclusion/exclusion) of subjects enrolled in drug clinical trials. The present invention provides a simple, rapid, non-invasive phenotype breath test for evaluating CYP2D6 activity in individual subjects.

IV. Preparation and Methods of the Invention

A. Isotope-labeled CYP2D6 Substrate Preparations of the Invention

The present invention provides preparations for easily determining and assessing the CYP2D6-related metabolic capacity in an individual mammalian subject. The preparations are useful for determining the CYP2D6-related metabolic behavior in a subject and easily assessing the metabolic capacity and identifying a clinical response and/or medical condition related to CYP2D6 activity in the subject. Specifically, the preparations of the invention are useful to determine and assess the CYP2D6-related metabolic capacity in an individual subject at the clinic setting (point of care) by measuring the metabolic behavior of a CYP2D6 enzyme substrate compound, in particular the excretion pattern of a metabolite of such a compound (including excretion amount, excretion rate, and change in the amount and rate with the lapse of time), in the subject.

A preparation useful in the methods of the present invention contains an isotopically labeled CYP2D6 substrate compound as an active ingredient. In one embodiment, the CYP2D6 substrate compound is a CYP2D6 substrate of Table 2 in which at least one of the carbon or oxygen atoms is labeled with an isotope and the preparation is capable of producing isotope labeled CO₂ after administration to a subject. The CYP2D6 substrate compound of the invention can be labeled in at least one position with ¹³C; ¹⁴C; and ¹⁸O. In a preferred embodiment, a CYP2D6 substrate compound is isotopically labeled with ¹³C such that the preparation is capable of producing stable ¹³CO₂ after administration to a subject. For example, breath tests utilizing dextromethorphan (DXM), tramadol, codeine, methacetin, aminopyrin, caffeine and erythromycin-¹³C as substrates are all dependent on N- or O-demethylation reactions and subsequently, the metabolic fate of the released methyl group through the body's one carbon pool ultimately to form ¹³CO₂ (or ¹⁴CO₂, depending on the isotope used) that is released in expired breath over time:

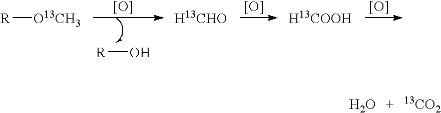

In a preferred embodiment, the CYP2D6 substrate compound is ¹³C-labeled DXM; ¹³C-labeled Tramadol; or ¹³C-labeled codeine and not limited to these substrates. A preparation of the invention may be formulated with a pharmaceutically acceptable carrier.

As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field. Supplementary active compounds can also be incorporated into the compositions.

The method for labeling a CYP2D6 substrate compound with an isotope is not limited and may be a conventional method (Sasaki, “5.1 Application of Stable Isotopes in Clinical Diagnosis”: Kagaku no Ryoiki (Journal of Japanese Chemistry) 107, “Application of Stable Isotopes in Medicine, Pharmacy, and Biology”, pp. 149-163 (1975), Nankodo: Kajiwara, RADIOISOTOPES, 41, 45-48 (1992)). Some isotopically labeled CYP2D6 substrate compounds are commercially available, and these commercial products are conveniently usable. For example, ¹³C-DXM and ¹³C-Tramadol substrates capable of producing ¹³CO₂ after administration to a subject are useful in the methods of the invention and are commercially available from Cambridge Isotope Laboratories, Inc. (Andover, Mass., USA).

A pharmaceutical composition of the invention is formulated to be compatible with its intended route of administration. Examples of routes of administration include parenteral, e.g., intravenous, intradermal, subcutaneous, oral (e.g., inhalation), transmucosal, and rectal administration. The preparation of the present invention may be in any form suitable for the purposes of the present invention. Examples of suitable forms include injections, intravenous injections, suppositories, eye drops, nasal solutions, and other parenteral forms; and solutions (including syrups), suspensions, emulsions, tablets (either uncoated or coated), capsules, pills, powders, subtle granules, granules, and other oral forms. Oral compositions generally include an inert diluent or an edible carrier.

The preparation of the present invention may consist substantially of the isotope-labeled CYP2D6 substrate compound as an active ingredient, but may be a composition further containing a pharmaceutically acceptable carrier or additive generally used in this field according to the form of the preparation (dosage form) (composition for determining CYP2D6 metabolic capacity), as long as the actions and effects of the preparation of the present invention are not impaired. In such a composition, the proportion of the isotope-labeled CYP2D6 substrate compound as an active ingredient is not limited and may be from about 0.1 wt % to about 99 wt % of the total dry weight of the composition. The proportion can be suitably adjusted within the above range.

When the isotope-labeled CYP2D6 substrate composition is formed into tablets, useful carriers include, but are not limited to, e.g., lactose, sucrose, sodium chloride, glucose, urea, starches, calcium carbonate, sodium and potassium bicarbonate, kaolin, crystalline cellulose, silicic acid, and other excipients; simple syrups, glucose solutions, starch solutions, gelatin solutions, carboxymethyl cellulose, shellac, methyl cellulose, potassium phosphate, polyvinyl pyrrolidone, and other binders; dry starches, sodium alginate, agar powder, laminaran powder, sodium hydrogencarbonate, calcium carbonate, polyoxyethylene sorbitan, fatty acid esters, sodium lauryl sulfate, stearic acid monoglyceride, starches, lactose, and other disintegrators; sucrose, stearic acid, cacao butter, hydrogenated oils, and other disintegration inhibitors; quaternary ammonium bases, sodium lauryl sulfate, and other absorption accelerators; glycerin, starches, and other humectants; starches, lactose, kaolin, bentonite, colloidal silicic acid, and other adsorbents; and purified talc, stearate, boric acid powder, polyethylene glycol, and other lubricants. Further, the tablets may be those with ordinary coatings (such as sugar-coated tablets, gelatin-coated tablets, or film-coated tablets), double-layer tablets, or multi-layer tablets.

When forming the composition for determining CYP2D6-related metabolic capacity into pills, useful carriers include, for example, glucose, lactose, starches, cacao butter, hydrogenated vegetable oils, kaolin, talc, and other excipients; gum arabic powder, tragacanth powder, gelatin, and other binders; and laminaran, agar, and other disintegrators. Capsules are prepared in a routine manner, by mixing the active ingredient according to the present invention with any of the above carriers and then filling the mixture into hardened gelatin capsules, soft capsules, or the like. Useful carriers for use in suppositories include, for example, polyethylene glycol, cacao butter, higher alcohols, esters of higher alcohols, gelatin, and semisynthetic glyceride.

An oral liquid solution is prepared in a routine manner, by mixing the active ingredient according to the present invention with any of carriers in common use. Specific examples of the oral liquid solution include a syrup preparation. The syrup preparation does not have to be liquid but may be a dry syrup preparation having a form of powder or granular.

When the preparation is prepared in the form of an injection, the injection solution, emulsion or suspension is sterilized and preferably isotonic with blood. Useful diluents for preparing the injection include, for example, water, ethyl alcohol, macrogol, propylene glycol, ethoxylated isostearyl alcohol, polyoxylated isostearyl alcohol, and polyoxyethylene sorbitan fatty acid esters. The injection may contain sodium chloride, glucose, or glycerin in an amount sufficient to make an isotonic solution. Also, an ordinary solubilizer, buffer, soothing agent or the like can be added to the injection.

Further, the preparation of the present invention in any of the above forms may contain a pharmaceutically acceptable additive, such as a color, preservative, flavor, odor improver, taste improver, sweetener, or stabilizer. The above carriers and additives may be used either singly or in combination. The amount of the isotope-labeled CYP2D6 substrate compound (active ingredient) per unit dose of the preparation of the present invention varies depending on the test sample and the kind of active ingredient used, and cannot be generally defined. A preferred amount is, for example, 1 to 300 mg/body per unit dose, although it is not limited thereto as long as the above condition is satisfied.

B. Methods of the Invention

A medical condition or clinical response related to CYP2D6 enzyme activity in a subject can be easily assessed using the methods of the present invention by administering an isotope-labeled CYP2D6 substrate compound to the subject and measuring the excretion pattern (including excretion amount, excretion rate, and change in the amount and rate with the lapse of time) of isotope-labeled CO₂ in the expired air. As such, the present invention provides methods to determine the clearance of an isotope-labeled CYP2D6 substrate compound to establish a more effective dosage regimen (formula, dose, number of doses, etc.) of the CYP2D6 substrate compound for individual subjects based on the CYP2D6 metabolic capacity in these subjects.

In some embodiments of the method, at least one CYP2D6 modulating agent is administered to a subject prior to administering a isotope-labeled CYP2D6 substrate compound. Such methods are useful to modulate (increase or decrease) CYP2D6 metabolic capacity in a subject. For example, administration of an inhibitor of CYP2D6 enzyme function is useful to decrease CYP2D6 metabolic capacity in a subject such that they display a PM or IM phenotype with respect to metabolism of CYP2D6 substrate. Alternatively, administration of an inducer of CYP2D6 enzyme is useful to increase CYP2D6 metabolic capacity in a subject such that they display a EM or UM phenotype with respect to metabolism of CYP2D6 substrate.

In one embodiment, the invention provides a method for determining CYP2D6 metabolic capacity, by administering an isotope-labeled CYP2D6 substrate preparation of the invention to a mammalian subject, and measuring the excretion pattern of an isotope-labeled metabolite excreted from the body. In one embodiment, the isotope-labeled metabolite is excreted from the body as stable isotope-labeled CO₂ in the expired air.

The isotope-labeled metabolite in the test sample can be measured and analyzed by a conventional analysis technique, such as liquid scintillation counting, mass spectroscopy, infrared spectroscopic analysis, emission spectrochemical analysis, or nuclear magnetic resonance spectral analysis, which is selected depending on whether the isotope used is radioactive or non-radioactive. The ¹³CO₂ can be measured by any method known in the art, such as any method that can detect the amount of exhaled ¹³CO₂. For example, ¹³CO₂ can be measured spectroscopically, such as by infrared spectroscopy. One exemplary device for measuring ¹³CO₂ is the UBiT.-IR300 infrared spectrometer, commercially available from Meretek (Denver, Colo., USA.). The subject, having ingested the ¹³C-labeled CYP2D6 substrate compound, can exhale into a breath collection bag, which is then attached to the UBiT-IR300. The UBiT-IR300 measures the ratio of ¹³CO₂ to ¹²CO₂ in the breath. By comparing the results of the measurement with that of a standard, or pre ¹³C-labeled CYP2D6 substrate ingestion breath the amount of exhaled ¹³CO₂ can be subsequently calculated. Alternatively, the exhaled ¹³CO₂ can be measured with a mass analyzer.

The preparation of the present invention is administered via the oral or parenteral route to a subject and an isotope-labeled metabolite excreted from the body is measured, so that the CYP2D6-related metabolic capacity (existence, nonexistence, or degree of CYP2D6-related medical condition, e.g., a metabolic disorder (decrease/increase)), in the subject can be determined from the obtained excretion pattern (the behavior of excretion amount and excretion rate with the lapse of time) of the isotope-labeled metabolite. The metabolite excreted from the body varies depending on the kind of the active ingredient used in the preparation. For example, when the preparation comprises isotope-labeled DXM as an active ingredient, the final metabolite is dextrorphan and isotope-labeled CO₂ (see generally, Example 1, infra). Preferably, the preparation comprises, as an active ingredient, an isotope-labeled CYP2D6 substrate compound that enables the excretion of isotope-labeled CO₂ in the expired air as a result of metabolism. Using such a preparation, the CYP2D6-related metabolic capacity (existence, nonexistence, or degree of CYP2D6-related metabolic disorder (decrease/increase)) in a subject can be determined from the excretion pattern (the behavior of excretion amount and excretion rate with the lapse of time) of isotope-labeled CO₂, which is obtained by administering the preparation to the subject via the oral or parenteral route and measuring isotope-labeled CO₂ excreted in the expired air.

In one embodiment, the invention provides a method for determining CYP2D6-related metabolic capacity in a mammalian subject, by administering an isotope labeled CYP2D6 substrate preparation of the invention to a subject, measuring the excretion pattern of an isotope-labeled metabolite excreted from the body, and assessing the obtained excretion pattern in the subject. In one embodiment of the method, an isotope-labeled CYP2D6 substrate preparation is administered to a mammalian subject, the excretion pattern of isotope-labeled CO₂ in the expired air is measured, and assessed. In one embodiment of the method, the excretion pattern of isotope-labeled CO₂ or a pharmacokinetic parameter obtained therefrom is compared with the corresponding excretion pattern or parameter in a healthy subject with a normal CYP2D6-metabolic capacity. That is, the CYP2D6-related metabolic capacity in a subject can be assessed by, for example, comparing the excretion pattern (the behavior of excretion amount or excretion rate with the lapse of time) of an isotope-labeled metabolite obtained by the above measurement, with the excretion pattern of the isotope-labeled metabolite in a reference standard, which is measured in the same manner. Further, in place of, or in addition to, the excretion pattern of an isotope-labeled metabolite, the area under the curve (AUC), excretion rate (in particular, initial excretion rate), maximum excretion concentration (Cₘₐₓ), slope of the δ¹³CO₂ as a function of time or percent dose recovery as a function of time, delta over baseline (DOB) at a particular timepoint or a similar parameter (preferably pharmacokinetic parameter) obtained from the excretion pattern (transition curve of the excretion amount) in the subject is compared with the corresponding parameter in reference standard. In one embodiment, the reference standard is the excretion pattern observed in a one or more healthy subject with normal metabolic activity.

In one embodiment, CYP2D6-related metabolic capacity is determined by an area under the curve (AUC), which plots the amount of exhaled ¹³CO₂ on the y-axis versus the time after the ¹³C-labeled CYP2D6 substrate is ingested. The area under the curve represents the cumulative δ¹³CO₂ recovered.

¹³CO₂ is also quantified as δ¹³CO₂ (a.k.a., DOB) according to the following equation:

δ¹³CO₂ equals (δ¹³CO₂ in sample gas minus δ¹³CO₂ in baseline sample before ingestion of ¹³C-labeled CYP2D6 substrate) where δ values are calculated (in) by=[(Rₛₐₘₚₗₑ/R_{standard})−1]×1000, and “R” is the ratio of the heavy to light isotope (¹³C/¹²C) in the sample or standard.

¹³CO₂ (or ¹⁴CO₂) and ¹²CO₂ in exhaled breath samples is measured by IR spectrometry using the UBiT-IR300 (Meretek Diagnostics, Lafayette, Colo.; ¹³CO₂ urea breath analyzer instruction manual. Lafayette, Colo.: Meretek Diagnostics; 2002; A1-A2). See Meretek Diagnostics, Inc. Meretek UBiT-IR300: ¹³CO₂ urea breath analyzer instruction manual. Lafayette, Colo.: Meretek Diagnostics; 2002; A1-A2.

The amount of ¹³CO₂ present in breath samples is expressed as delta over baseline (DOB) that represents a change in the ¹³CO₂/¹²CO₂ ratio of breath samples collected before and after ¹³C-labeled CYP2D6 substrate compound ingestion.

$$DOB = \frac{{}^{13} {CO}_{2}}{{}^{12} {CO}_{{2}_{{}_{sample}^{Post dose}}}} - \frac{{}^{13} {CO}_{2}}{{}^{12} {CO}_{{2}_{{}_{sample}^{Pre dose}}}}$$

The amount of ¹³C-labeled CYP2D6 substrate compound absorbed and released into the breath as ¹³CO₂ is determined for each time point using the equation described by Amarri. Amarri et al., Clin Nutr. 14: 149-54 (1995). These results are expressed as percentage dose recovery (PDR).

The PDR is calculated using the formula:

$$\frac{\frac{( {δ}_{t}^{13} - {δ}_{0}^{13} ) + ( {δ}_{t + 1}^{13} - {δ}_{0}^{13} )}{2} \times ( {t}_{+ 1} - t ) \times {R}_{PDB} \times {10}^{- 3} \times C}{\frac{mg substrate}{mol . wt .} \times \frac{P \times n}{100}} \times 100 \%$$

where ¹³δ=[R_{S}/R_{PDB)}−1]×10³

Rₛ=¹³C: ¹²C in the sample

R_{PDB}=¹³C: ¹²C in PDB (international standard PeeDeeBelemnite)=0.0112372)

P is the atom % excess

n is the number of labeled carbon positions

δₜ, δₜ₊₁, δ₀ are enrichments at times t, t₊₁ and predose respectively

C is the CO₂ production rate (C=300 [mmol/h]*BSA

BSA=w^{0.5378}*h^{0.3963}*0.024265 (Body Surface Area)

w: Weight (kg)

h: Height (cm)

Cₘₐₓ is the highest value of DOB from the breath curve following ¹³C-labeled CYP2D6 substrate compound.

As noted above, the invention provides a method for determining the existence, nonexistence, or degree of CYP2D6-related metabolic disorder (i.e., a medical condition) in a mammalian subject by administering a preparation of the invention to a mammalian subject, measuring the excretion pattern of an isotope-labeled metabolite excreted from the body, and assessing the obtained excretion pattern in the subject. In a preferred embodiment of the method, the isotope-labeled metabolite is excreted from the body as stable isotope-labeled CO₂ in the expired air.

In one embodiment, the invention provides a method for selecting a prophylactic or therapeutic treatment for a subject by (a) determining the phenotype of the subject; (b) assigning the subject to a subject class based on the phenotype of the subject; and (c) selecting a prophylactic or therapeutic treatment based on the subject class, wherein the subject class (subject class I) comprises two or more individuals who display a level of CYP2D6-related metabolic activity that is at least about 10% lower than a reference standard level of CYP2D6-related metabolic activity. In one embodiment of the method, the subject class (subject class II) comprises two or more individuals who display a level of CYP2D6-related metabolic activity that is at least about 10% higher than a reference standard level of CYP2D6-related metabolic activity. In one embodiment of the method, the subject class (subject class III) comprises two or more individuals who display a level of CYP2D6-related metabolic activity within at least about 10% of a reference standard level of CYP2D6-related metabolic activity. The subject with PM or IM phenotype may be assigned to the subject class I, and the subject with EM or UM phenotype may be assigned to the subject class III or II, respectively.

The therapeutic treatment selected can be administering a drug, selecting a drug dosage, and selecting the timing of a drug administration.

In one embodiment, the invention provides a method for evaluating CYP2D6-related metabolic capacity, by administering a ¹³C-labeled CYP2D6 substrate compound to a mammalian subject; measuring ¹³CO₂ exhaled by the subject; and determining CYP2D6-related metabolic capacity from the measured ¹³CO₂. In one embodiment of the method, the ¹³C-labeled substrate is selected from the group consisting of: a ¹³C-labeled DXM; ¹³C-labeled Tramadol; and ¹³C-labeled codeine. In one embodiment of the method, the ¹³C-labeled substrate compound is administered non-invasively. In one embodiment of the method, the ¹³C-labeled substrate compound is administered intravenously or by oral route. In one embodiment of the method, the exhaled ¹³CO₂ is measured spectroscopically. In one embodiment of the method, the exhaled ¹³CO₂ is measured by infrared spectroscopy. In another embodiment of the method, the exhaled ¹³CO₂ is measured with a mass analyzer. In one embodiment of the method, the exhaled ¹³CO₂ is measured over at least three time periods to generate a dose response curve, and the CYP2D6-related metabolic activity is determined from the area under the curve. In one embodiment of the method, the exhaled ¹³CO₂ is measured over at least two different dosages of the ¹³C-labeled CYP2D6 substrate compound. In one embodiment of the method, the exhaled ¹³CO₂ is-measured during at least the following time points: t₀, a time prior to ingesting the ¹³C-labeled CYP2D6 substrate compound; t₁, a time after the ¹³C-labeled CYP2D6 substrate compound has been absorbed in the bloodstream of the subject; and t₂, a time during the first elimination phase. In one embodiment of the method, CYP2D6-related metabolic capacity is determined from as the a slope of δ¹³CO₂ at time points t₁ and t₂ calculated according to the following equation: slope=[(δ¹³CO₂)₂−(δ¹³CO₂)₁]/(t₂−t₁)- wherein δ¹³CO₂ is the amount of exhaled ¹³CO₂. In another embodiment of the invention, at least one CYP2D6 modulating agent is administered to the subject before administrating a ¹³C-labeled CYP2D6 substrate compound. The CYP2D6 modulating agent used in the method of the invention can be an inhibitor of CYP2D6 enzyme activity or and inducer of CYP2D6 enzyme activity. CYP2D6 inhibitors summarized in Table 3 are useful in the method of the invention. Likewise, compounds that induce CYP2D6 include, e.g., Ritonavir; Amiodarone; Quinidine; Paroxetine; Cimetidine; Fluoxetine; dexamethasone; and Rifampin, are also useful in the method of the invention. The CYP2D6. can be administered to a subject in any suitable dose or time interval prior to administration of the ¹³C-labeled CYP2D6 substrate compound to give the desired inhibition or induction/activation of CYP2D6 metabolic capability in a subject.

In one embodiment, the invention provides a method of selecting a mammalian subject for inclusion in a clinical trial for determining the efficacy of a compound to prevent or treat a medical condition, comprising the steps of: (a) administering a ¹³C-labeled cytochrome P450 2D6 isoenzyme substrate compound to the subject; (b) measuring the excretion pattern of an isotope-labeled metabolite excreted from the body of the subject; (c) comparing the obtained excretion pattern in the subject to a reference standard excretion pattern; and (d) selecting to include the subject in the clinical trial, wherein a similarity in the excretion pattern of the subject is similar to the excretion pattern of the standard gene excretion pattern.

The method of the present invention can be non-invasive, only requiring that the subject perform a breath test. The present test does not require a highly trained technician to perform the test. The test can be performed at a general practitioners office, where the analytical instrument (such as, e.g., a UBiT-IR300) is installed. Alternatively, the test can be performed at a user's home where the home user can send breath collection bags to a reference lab for analysis.

Another embodiment of the invention provides a kit for determining CYP2D6-related metabolic capacity. The kit can include ¹³C-labeled CYP2D6 substrate compound (e.g., ¹³C-labeled DXM; ¹³C-labeled Tramadol; and ¹³C-labeled codeine) and instructions provided with the substrate that describe how to determine CYP2D6-related metabolic capacity in a subject. The ¹³C-labeled CYP2D6 substrate compound can be supplied as a tablet, a powder or granules, a capsule, or a solution. The instructions can describe the method for CYP2D6-related metabolic capacity by using the area under the curve, or by the slope technique, or other pharmacokinetic parameters as described above. The kit can include at least three breath collection bags. In one embodiment of the kit, the kit further comprises of a CYP2D6 modulating agent.

C. Select Clinical Applications of the Method of the Invention
i. Correlating a Subject to a Standard Reference Population

One aspect of the invention relates to diagnostic assays for determining CYP2D6-related metabolic capacity, in the context of a biological sample (e.g., expired air) to thereby determine whether an individual is afflicted with a disease or disorder, or is at risk of developing a disorder, associated with aberrant CYP2D6 expression or activity. To deduce a correlation between clinical response to a treatment and a gene expression pattern or phenotype, it is necessary to obtain data on the clinical responses exhibited by a population of individuals who received the treatment, i.e., a clinical population. This clinical data may be obtained by retrospective analysis of the results of a clinical trial(s). Alternatively, the clinical data may be obtained by designing and carrying out one or more new clinical trials. The analysis of clinical population data is useful to define a standard reference population(s) which, in turn, are useful to classify subjects for clinical trial enrollment or for selection of therapeutic treatment. It is preferred that the subjects included in the clinical population have been graded for the existence of the medical condition of interest, e.g., CYP2D6 PM phenotype, CYP2D6 IM phenotype, CYP2D6 EM phenotype, or CYP2D6 UM phenotype. Grading of potential subjects can include, e.g., a standard physical exam or one or more tests such as the breath test of the present invention. Alternatively, grading of subjects can include use of a gene expression pattern, e.g., CYP2D6 allelic variants (see Table 4). For example, gene expression pattern is useful as grading criteria where there is a strong correlation between gene expression pattern and phenotype or disease susceptibility or severity. ANOVA is used to test hypotheses about whether a response variable is caused by, or correlates with, one or more traits or variables that can be measured. Such standard reference population comprising subjects sharing gene expression pattern profile and/or phenotype characteristic(s), are useful in the methods of the present invention to compare with the measured level of CYP2D6-related metabolic capacity or CYP2D6 metabolite excretion pattern in a given subject. In one embodiment, a subject is classified or assigned to a particular genotype group or phenotype class based on similarity between the measured expression pattern of CYP2D6 metabolite and the expression pattern of CYP2D6 metabolite observed in a reference standard population. The method of the present invention is useful as a diagnostic method to identify an association between a clinical response and a genotype or haplotype (or haplotype pair) for the CYP2D6 gene or a CYP2D6 phenotype. Further, the method of the present invention is useful to determine those individuals who will or will not respond to a treatment, or alternatively, who will respond at a lower level and thus may require more treatment, i.e., a greater dose of a drug.
ii. Monitoring Clinical Efficacy

The method of the present invention is useful to monitoring the influence of agents (e.g., drugs, compounds) on the expression or activity of CYP2D6-related metabolic capability and can be applied in basic drug screening and in clinical trials. For example, the effectiveness of an agent determined by a CYP2D6 phenotype assay of the invention to increase CYP2D6-related metabolic activity can be monitored in clinical trials of subjects exhibiting decreased CYP2D6-related metabolic capability. Alternatively, the effectiveness of an agent determined by a CYP2D6 phenotype assay of the invention to CYP2D6-related metabolic activity can be monitored in clinical trials of subjects exhibiting increased CYP2D6-related metabolic capacity.

Alternatively, the effect of an agent on CYP2D6-related metabolic capability during a clinical trial can be measured using the CYP2D6 phenotype assay of the present invention. In this way, the CYP2D6 metabolite expression pattern measured using the method of the present invention can serve as a benchmark, indicative of the physiological response of the subject to the agent. Accordingly, this response state of a subject may be determined before, and at various points during treatment of the individual with the agent.

The following Examples are presented in order to more fully illustrate the preferred embodiments of the invention. These Examples should in no way be construed as limiting the scope of the invention, as defined by the appended claims.

### EXAMPLES

### Example 1

### Classification of Human Subject by Dextramethorphan (DXM) Metabolic Capacity Using the ¹³CO₂ Breath Test Method of the Invention

The semisynthetic narcotic DXM is an antitussive found in a variety of over-the-counter medicines useful to relieve a nonproductive cough caused by a cold, the flu, or other conditions. DXM acts centrally to elevate the threshold for coughing. At the doses recommended for treating coughs (⅙ to ⅓ ounce of medication, containing 15 mg to 30 mg DXM), the drug is safe and effective. At much higher doses (four or more ounces), DXM produces disassociative effects similar to those of PCP and ketamine. DXM metabolism is genetically polymorphous, similar to the codeine metabolism. CYP2D6 mediates the O-demethylation of DXM-O-¹³CH₃ as detailed below.

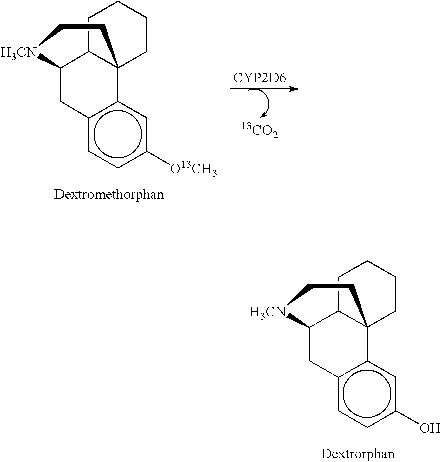

In addition to genetic factors, the apparent phenotype of an individual subject and overall significance of CYP2D6 in the biotransformation of a given substrate is influenced by the quantitative importance of alternative metabolic routes (Abdel-Rahman et al., Drug Metab. Disposit., 27(7): 770-775 (1999)). For example, agents that are preferentially metabolized by CYP2D6, pharmacologic inhibitors can modify enzyme activity such that the magnitude of change in substrate metabolism may mimic that of genetically determined poor metabolizers (i.e., an apparent change in phenotype from an extensive metabolizer to a poor metabolizer). With inhibitors of CYP2D6, the metabolism of coadministered CYP2D6 substrates may be significantly altered in close to 93% of the population classified as extensive metabolizers (Brosen et al., Eur. J. Clin. Invest., 36: 537-547 (1989)). Such interactions may decrease the efficacy of a prodrug requiring metabolic conversion to its active moiety or, alternately, may result in toxicity for CYP2D6 substrates that have a narrow therapeutic index. Non-invasive diagnostic/theranostic tests, e.g., breath tests, are useful to assess the CYP2D6 metabolic status of an individual subject.

The present studies employed the ¹³CO₂ breath test method of the present invention to classify individual human subjects (i.e., Volunteers 1 and 2) by their ability to metabolize DXM-O-¹³CH₃. Briefly, following an 8-12 h fast normal human subjects ingested 2 Alka seltzer Gold tablets (Bayer Healthcare). The tablets suppress heartburn and/or gastric hyperacidity, and each tablet comprises 1000 mg of citric acid, 344 mg of potassium bicarbonate, 1050 mg of sodium bicarbonate (heat-treated), 135 mg of potassium, 309 mg of sodium, and other components such as magnesium stearate and mannitol. Since drug absorption is slow in subjects with heartburn and/or gastric hyperacid, such subjects, even if having normal metabolism, may be misdiagnosed as having slow or no metabolism of the test drug (as being EM, IM or PM). Thus, the tablets are administered in order to eliminate “individual differences in absorption” occurring when orally administering a ¹³C-labeled CYP2D6 substrate compound (e.g., DXM).

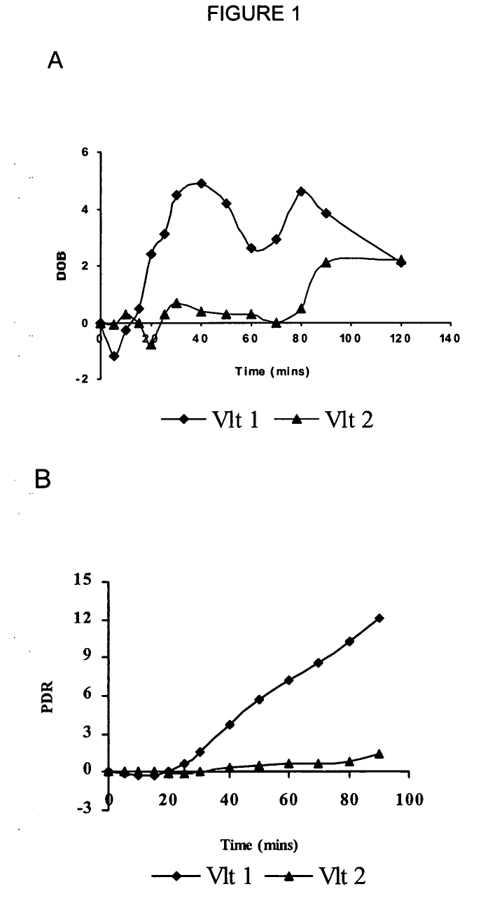

Thirty minutes after ingesting the Alka seltzer Gold tablets the subjects ingested 75 mg of DXM-O—¹³CH₃. Breath samples were collected prior to drug ingestion and then at 5 min time points up to 30 min, at 10 min intervals to 90 min, and 30 min intervals thereafter to 120 min after ingestion of DXM-O—¹³CH₃. The breath curves (DOB versus Time (Panel A) and PDR versus Time (Panel B)) for two volunteers for the DXM-O—13CH₃ breath test are depicted in FIG. 1. Volunteer 1 was an extensive DXM metabolizer (EM) with the CYP2D6*1/*1 genotype. The YP2D6*1/*1 genotype has any of alleles CYP2D6*1A to CYP2D6*1XN in homozygous or heterozygous form, and has normal DXM metabolic capacity based on normal CYP2D6 enzyme activity. Volunteer 2 was a poor DXM metabolizer (PM) with a *5 allele, gene deletion. (Courtesy of Leeder et al., CMH, Kansas City, Mo.). That is, Volunteer 2 is deficient in the total CYP2D6 genome, and in Volunteer 2, CYP2D6 enzyme is not synthesized at all (no DXM metabolic capacity) (corresponding to CYD2D6*5 in Table 4). The present studies demonstrate that either DOB or PDR values at a specific time point are useful to differentiate EM's (two or more alleles) from PM's (zero or one allele).

The DXM-O-¹³CH₃ phenotyping procedure with a ¹³CO₂ breath test has several potential advantages over existing phenotyping methods, as mass spectrometry detection can be replaced by infrared spectrometry. In addition to the safety and demonstrated utility of DXM as a probe for CYP2D6 activity, the breath test affords phenotype determinations within a shorter time frame (1 h or less after DXM administration) and directly in physicians' offices or other healthcare settings using relatively cheap instrumentation (UBiT-IR₃₀₀ IR spectrophotometer; Meretek).

### Example 2

### Classification of Human Subjects by Tramadol Metabolic Capacity Using the ¹³CO₂ Breath Test Method of the Invention

(+/−)-Tramadol, a synthetic analogue of codeine, is a central analgesic with a low affinity for select receptors, e.g., Mu opioid receptor. (+/−)-Tramadol is a racemic mixture of two enantiomers, each displaying differing affinities for various receptors. (+)-Tramadol is a receptive agonist of Mu receptors and preferentially inhibits seratonin reuptake, where as (−)-tramadol mainly inhibits norepinephrine reuptake. The action of these two enantiomers is both complimentary and synergistic and results in the analgesic affect of (+/−)-tramadol.

(+/−)-Tramadol is transformed in mammals to an O-demethylated metabolite called “M1”, i.e., O-desmethyl tramadol. The M1 metabolite of tramadol, shows a higher affinity for opioid receptors than the parent drug. The rate of production of the M1 derivative is influenced by the enzymatic action of CYP2D6. CYP2D6 converts (+/−)-tramadol to M1 with the concomitant release of carbon dioxide which can be excreted from the body of a subject in expired air.

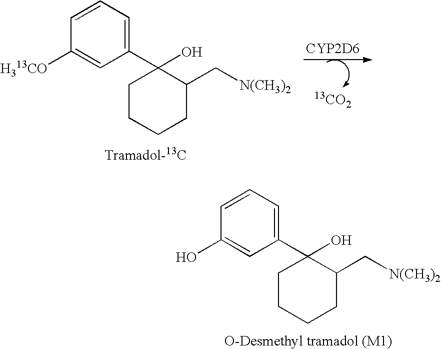

As noted above, in addition to genetic factors, the apparent phenotype of an individual subject and overall significance of CYP2D6 in the biotransformation of a given substrate is influenced by the quantitative importance of alternative metabolic routes (Abdel-Rahman et al., Drug Metab. Disposit., 27(7): 770-775 (1999)). Such interactions may decrease the efficacy of a prodrug requiring metabolic conversion to its active moiety or, alternately, may result in toxicity for CYP2D6 substrates that have a narrow therapeutic index. (+/−)-Tramadol is an agent effective for moderate to severe pain, in adults and children. Potential problems include CYP2D6 deficiency, which may have clinical consequences (about 30% of analgesia is from M1 metabolite). (+/−)-Tramadol may be more effective in extensive metabolizers. Non-invasive diagnostic/theranostic tests, e.g., breath tests, are useful to assess the CYP2D6 metabolic status of an individual subject.

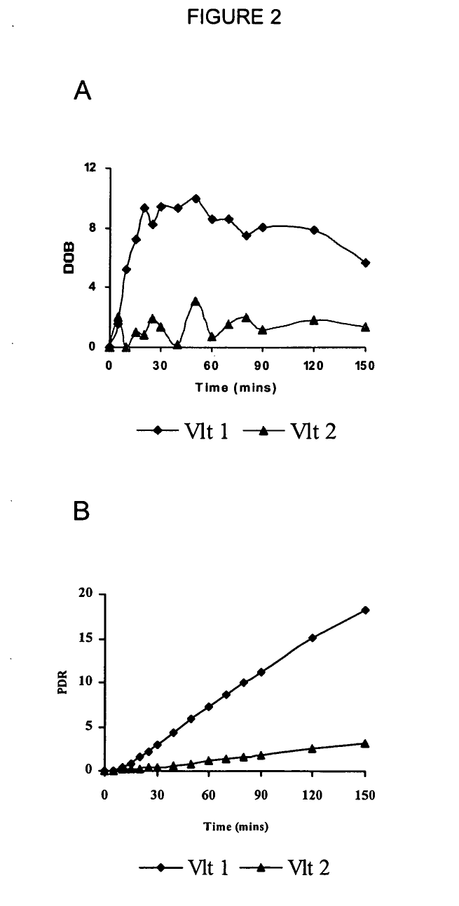

The present studies employed the ¹³CO₂ breath test method of the present invention to classify individual human subjects (i.e., Volunteers 1 and 2) by their ability to metabolize (+/−)-tramadol-O-¹³CH₃. Briefly, following an 8-12 h fast normal human subjects ingested 2 Alka seltzer Gold tablets. Thirty minutes after ingesting the Alka seltzer Gold tablets the subjects ingested 75 mg of (+/−)-tramadol-O—¹³CH₃ (˜1.5 mg/kg body weight). Breath samples were collected prior to ingestion of (+/−)-tramadol-O-¹³CH₃ and then at 5 min intervals to 30 min, at 10 min intervals to 90 min, and at 30 min intervals thereafter to 150 min after isotope ingestion. The breath curves (DOB versus Time (Panel A) and PDR versus Time (Panel B) for two volunteers for the (+/−)-tramadol-O—¹³CH₃ breath test are depicted in FIG. 2. Volunteer 1 was an extensive (+/−)-tramadol metabolizer (EM) with the CYP2D6*1/*1 genotype. Volunteer 2 was a poor (+/−)-tramadol metabolizer (PM) with a *5 allele, gene deletion. (Courtesy of Leeder et al., CMH, Kansas City, Mo.). The present studies demonstrate that either DOB or PDR values at a specific time point are useful to differentiate EM's (two or more alleles) from PM's (zero or one allele).

The (+/−)-tramadol phenotyping procedure with a ¹³CO₂ breath test has several potential advantages over existing phenotyping methods, as mass spectrometry detection can be replaced by infrared spectrometry. In addition to the safety and demonstrated utility of (+/−)-tramadol as a probe for CYP2D6 activity, the breath test affords phenotype determinations within a shorter time frame (one hour or less after (+/−)-tramadol administration) and directly in physicians' offices or other healthcare settings using relatively cheap instrumentation (UBiT-IR₃₀₀ IR spectrophotometer; Meretek).

### Example 3

### Breath Test Procedure

In one embodiment of the breath test procedure of the invention, ¹³C-labeled CYP2D6 substrate compound (0.1 mg-500 mg) is ingested by a subject after overnight fasting (8-12 h), over a time period of approximately 10-15 seconds. Breath samples are collected prior to ingestion of ¹³C-labeled CYP2D6 substrate compound and then at 5 min intervals to 30 min, at 10 minute intervals to 90 min, and at 30 min intervals thereafter to 150 min after isotope-labeled substrate ingestion. The breath samples are collected by having the subject momentarily hold their breath for 3 seconds prior to exhaling into a sample collection bag. The breath samples are analyzed on a UBiT IR-300 spectrophotometer (Meretek, Denver, Colo.) to determine the ¹³CO₂/¹²CO₂ ratio in expired breath, or sent to a reference lab.

### Example 4

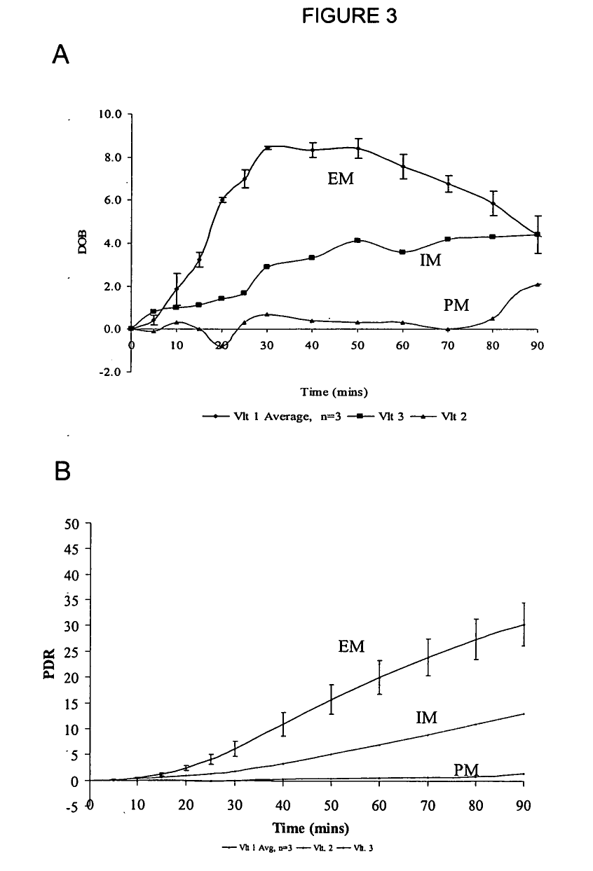

In one embodiment of the breath test, Alka seltzer tablet dissolved in water is ingested 15-30 minutes prior to ingestion of another Alka Seltzer tablet dissolved in water along with DXM-O—¹³CH₃ (75 mg) by three subjects (Volunteers 1, 2 and 3) after an overnight fast (8-12 h). Breath samples are collected prior to ingestion and at 5, 10, 15, 20, 25, 30 min, then at 10 minutes intervals to 60 min, and at 90 min after DXM-O—¹³CH₃ ingestion. The breath curves (DOB versus Time (Panel A) and PDR versus Time (Panel B) for three volunteers for the DXM-O—¹³CH₃ breath test are depicted in FIG. 3. Volunteers 1, 2 and 3 were an extensive DXM metabolizer (EM) with the CYP2D6*1/*1 genotype, a poor DXM metabolizer (PM) with a *5 allele, gene deletion (CYP2D6*5 genotype), and an intermediate metabolizer (IM; CYP2D6*1/*4 genotype), respectively. Volunteer 3 is of a genotype (CYP2D6*1/*4 genotype) having one of the alleles CYP2D6*1A to CYP2D6*1XN shown in Table 4 and one of the alleles CYP2D6*4A to CYP2D6*4X2 shown in Table 4. In CYP2D6, allele*1 has normal CYP2D6 activity, whereas allele*4 has lost its activity, and therefore CYP2D6*1/*4 as a whole has only half the activity of CYP2D6.

The present studies demonstrate that either DOB or PDR values at a specific time point are useful to differentiate among EM's, IM's and PM's. In other words, the Examples demonstrate that the breath test of the present invention can be applied to the diagnosis of subjects (IM) having a CYP2D6 enzyme activity level between EM and PM.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

We claim:

1. A preparation for determining cytochrome P450 2D6 isoenzyme-related metabolic capacity, comprising as an active ingredient a cytochrome P450 2D6 isoenzyme substrate compound in which at least one of the carbon or oxygen atoms is labeled with an isotope, wherein the preparation is capable of producing isotope-labeled CO₂ after administration to a mammalian subject.

2. The preparation according to claim 1, wherein the isotope is at least one isotope selected from the group consisting of: ¹³C; ¹⁴C; and ¹⁸O.

3. A method for determining cytochrome P450 2D6 isoenzyme-related metabolic capacity, comprising the steps of administering a preparation according to claim 1 to a mammalian subject, and measuring the excretion pattern of an isotope-labeled metabolite excreted from the body of the subject.

4. The method according to claim 3, wherein the isotope-labeled metabolite is excreted from the body as isotope-labeled CO₂ in the expired air.

5. A method for determining cytochrome P450 2D6 isoenzyme-related metabolic capacity in a mammalian subject, comprising the steps of administering a preparation of claim 1 to the subject, measuring the excretion pattern of an isotope-labeled metabolite excreted from the body of the subject, and assessing the obtained excretion pattern in the subject.

6. The method according to claim 5, comprising the steps of administering a preparation of claim 1 to a mammalian subject, measuring the excretion pattern of isotope-labeled CO₂ in the expired air, and assessing the obtained excretion pattern of CO₂ in the subject.

7. The method according to claim 5, comprising the steps of administering a preparation of claim 1 to a mammalian subject, measuring the excretion pattern of an isotope-labeled metabolite, and comparing the obtained excretion pattern in the subject or a pharmacokinetic parameter obtained therefrom with the corresponding excretion pattern or parameter in a healthy subject with a normal cytochrome P450 2D6 isoenzyme-related metabolic capacity.

8. A method for determining the existence, nonexistence, or degree of cytochrome P450 2D6 isoenzyme-related metabolic disorder in a mammalian subject, comprising the steps of administering a preparation of claim 1, to the subject, measuring the excretion pattern of an isotope-labeled metabolite excreted from the body of the subject, and assessing the obtained excretion pattern in the subject.

9. A method for selecting a prophylactic or therapeutic treatment for a subject, comprising:
(a) determining the phenotype of the subject;
(b) assigning the subject to a subject class based on the phenotype of the subject; and
(c) selecting a prophylactic or therapeutic treatment based on the subject class, wherein the subject class comprises two or more individuals who display a level of cytochrome P450 2D6 isoenzyme-related metabolic capacity that is at least about 10% lower than a reference standard level of cytochrome P450 2D6 isoenzyme-related metabolic capacity.

10. The method according to claim 9, wherein the subject class comprises two or more individuals who display a level of cytochrome P450 2D6 isoenzyme-related metabolic capacity that is at least about 10% higher than a reference standard level of cytochrome P450 2D6 isoenzyme-related metabolic capacity.

11. The method according to claim 9, wherein the subject class comprises two or more individuals who display a level of cytochrome P450 2D6 isoenzyme-related metabolic capacity within at least about 10% of a reference standard level of cytochrome P450 2D6 isoenzyme-related metabolic capacity.

12. The method according to claim 9, wherein the treatment is selected from administering a drug, selecting a drug dosage, and selecting the timing of a drug administration.

13. A method for evaluating cytochrome P450 2D6 isoenzyme-related metabolic capacity, comprising the steps of: administering a ¹³C-labeled cytochrome P450 2D6 isoenzyme substrate compound to a mammalian subject; measuring ¹³CO₂ exhaled by the subject; and determining cytochrome P450 2D6 isoenzyme-related metabolic capacity from the measured ¹³CO₂.

14. The method according to claim 13, wherein the ¹³C-labeled cytochrome P450 2D6 isoenzyme substrate compound is selected from the group consisting of: a ¹³C-labeled dextromethorphan; ¹³C-labeled tramadol; and ¹³C-labeled codeine.

15. The method according to claim 13, wherein the ¹³C-labeled cytochrome P450 2D6 isoenzyme substrate compound is administered non-invasively.

16. The method according to claim 13, wherein the ¹³C-labeled cytochrome P450 2D6 isoenzyme substrate compound is administered intravenously or orally.

17. The method according to claim 13, wherein the exhaled ¹³CO₂ is measured spectroscopically.

18. The method according to claim 13, wherein the exhaled ¹³CO₂ is measured by infrared spectroscopy.

19. The method according to claim 13, wherein the exhaled ¹³CO₂ is measured with a mass analyzer.

20. The method according to claim 13, wherein the exhaled ¹³CO₂ is measured over at least three time periods to generate a dose response curve, and the cytochrome 2D6 isoenzyme-related metabolic activity is determined from the area under the curve.

21. The method according to claim 20, wherein the exhaled ¹³CO₂ is measured over at least two different dosages of the ¹³C-labeled cytochrome P450 2D6 isoenzyme substrate compound.

22. The method according to claim 13, wherein the exhaled ¹³CO₂ is measured over at least three time periods to calculate a delta over baseline (DOB), and the cytochrome 2D6 isoenzyme-related metabolic activity is determined from the DOB.

23. The method according to claim 22, wherein the exhaled ¹³CO₂ is measured over at least two different dosages of the ¹³C-labeled cytochrome P450 2D6 isoenzyme substrate compound.

24. The method according to claim 13, wherein the exhaled ¹³CO₂ is measured over at least three time periods to calculate a percentage dose recovery (PDR), and the cytochrome 2D6 isoenzyme-related metabolic activity is determined from the PDR.

25. The method according to claim 24, wherein the exhaled ¹³CO₂ is measured over at least two different dosages of the ¹³C-labeled cytochrome P450 2D6 isoenzyme substrate compound.

26. The method according to claim 13, wherein the exhaled ¹³CO₂ is measured during at least the following time points: t₀, a time prior to ingesting the ¹³C-labeled cytochrome P450 2D6 isoenzyme substrate compound; t₁, a time after the ¹³C-labeled cytochrome P450 2D6 isoenzyme substrate compound has been absorbed in the bloodstream of the subject; and t₂, a time during the first elimination phase.

27. The method according to claim 26, wherein the cytochrome P450 2D6 isoenzyme-related metabolic capacity is determined from as the a slope of δ¹³CO₂ at time points t₁ and t₂ calculated according to the following equation: slope=[(δ¹³CO₂)₂—(δ¹³CO₂)₁]/(t₂−t₁)- wherein δ¹³CO₂ is the amount of exhaled ¹³CO₂.

28. The method according to claim 13, wherein a at least one cytochrome P450 2D6 isoenzyme modulating agent is administered to the subject before administrating a ¹³C-labeled cytochrome P450 2D6 isoenzyme substrate compound.

29. The method according to claim 28, wherein the cytochrome P450 2D6 modulating agent is a cytochrome P450 2D6 inhibitor.

30. The method according to claim 28, wherein the cytochrome P450 2D6 modulating agent is a cytochrome P450 2D6 inducer.

31. A method of selecting a mammalian subject for inclusion in a clinical trial for determining the efficacy of a compound to prevent or treat a medical condition, comprising the steps of:
(a) administering a ¹³C-labeled cytochrome P450 2D6 isoenzyme substrate compound to the subject;
(b) measuring a metabolite excretion pattern of an isotope-labeled metabolite excreted from the body of the subject; and
(c) comparing the obtained metabolite excretion pattern in the subject to a reference standard excretion pattern;
(d) classifying the subject according to a metabolic phenotype selected from the group consisting of: poor metabolizer, intermediate metabolizer, extensive metabolizer, and ultrarapid metabolizer, based on the obtained metabolite excretion pattern; and
(e) selecting the subject classified as extensive metabolizer in step (d) for inclusion in the clinical trial.

32. The method according to claim 31, wherein the isotope labeled metabolite excreted from the body of the subject is isotope-labeled CO₂ in the expired air.

33. A kit comprising: a ¹³C-labeled cytochrome P450 2D6 isoenzyme substrate compound; and instructions provided with the substrate that describe how to determine ¹³C-labeled cytochrome P450 2D6 isoenzyme substrate compound metabolism in a subject.

34. The kit according to claim 33, further comprising at least three breath collection bags.

35. The kit of according to claim 33, further comprising a cytochrome P450 2D6 modulating agent.
